(19) United States
(12) Patent Application Publication
(10) Pub. No.: US 2022/0403458 A1
(43) Pub. Date: Dec. 22, 2022

(54) METHODS TO GENERATE CIRCULARIZABLE PROBES IN SITU

(71) Applicant: 10x Genomics, Inc., Pleasanton, CA (US)

(72) Inventor: Felice Alessio BAVA, Rome (IT)

(21) Appl. No.: 17/843,758

(22) Filed: Jun. 17, 2022

Related U.S. Application Data
(60) Provisional application No. 63/213,133, filed on Jun. 21, 2021.

(51) Int. Cl. C12Q 1/6841; C12Q 1/682; C12Q 1/6844; C12P 19/34

(52) U.S. Cl. CPC C12Q 1/6841; C12Q 1/682; C12Q 1/6844; C12P 19/34

(57) ABSTRACT

The present disclosure relates in some aspects to methods, probes, kits, and compositions for analysis of a target nucleic acid, such as in situ generation of a circular probe for detection of a target nucleic acid in a tissue sample.

### CROSS-REFERENCE TO RELATED APPLICATION

This application claims priority to U.S. Provisional Patent Application No. 63/213,133, filed Jun. 21, 2021, entitled “METHODS TO GENERATE CIRCULARIZABLE PROBES IN SITU,” which is herein incorporated by reference in its entirety for all purposes.

### FIELD

The present disclosure relates in some aspects to methods, probes, kits, and compositions for analysis of a target nucleic acid, such as in situ generation of a circular probe for detection of a target nucleic acid in a tissue sample.

### BACKGROUND

Methods are available for analyzing nucleic acids present in a biological sample, such as a cell or a tissue. For instance, advances in single molecule fluorescent in situ hybridization (smFISH) have enabled nanoscale-resolution imaging of RNA in cells and tissues. However, length limits of oligonucleotide synthesis limit the production of probes containing higher numbers of barcode sequences. Improved methods and probes for analyzing nucleic acids present in a biological sample are needed. Provided herein are methods and compositions that address such and other needs.

### BRIEF SUMMARY

In some aspects, provided herein is a method for generating a circular probe, comprising: (a) contacting a target nucleic acid with a first probe, one or more splints, and a plurality of oligonucleotides, wherein: (i) the first probe comprises hybridization regions HR1, HR2, and HR3; (ii) the target nucleic acid comprises a hybridization region HR2′ that hybridizes to HR2; and (iii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints; and (b) ligating HR1 to a first adjacent oligonucleotide of the plurality of oligonucleotides, adjacent oligonucleotides of the plurality of oligonucleotides to one another, and HR3 to a second adjacent oligonucleotide of the plurality of oligonucleotides using the one or more splints as templates, thereby forming a circular probe that is hybridized to the target nucleic acid.

In some embodiments, the target nucleic acid can be contacted with the first probe, one or more splints, and plurality of oligonucleotides simultaneously or sequentially.

In some aspects, provided herein is a method for generating a circular probe, comprising: (a) contacting a target nucleic acid with a first probe, a second probe, one or more splints, and a plurality of oligonucleotides, wherein: (i) the first probe comprises hybridization regions HR1 and HR2a; (ii) the second probe comprises hybridization regions HR2b and HR3; (iii) the target nucleic acid comprises adjacent hybridization regions HR2a′ and HR2b′ that hybridize to HR2a and HR2b, respectively; and (iv) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints; and (b) ligating (i) HR1 to a first adjacent oligonucleotide of the plurality of oligonucleotides, adjacent oligonucleotides of the plurality of oligonucleotides to one another, and HR3 to a second adjacent oligonucleotide of the plurality of oligonucleotides using the one or more splints as templates, and (ii) HR2a to HR2b using the target nucleic acid as a template, thereby forming a circular probe that is hybridized to the target nucleic acid.

In some embodiments, the target nucleic acid can be contacted with the first probe, second probe, one or more splints, and plurality of oligonucleotides simultaneously or sequentially.

In some aspects, provided herein is a method for generating a circular probe, comprising: (a) contacting a target nucleic acid with a target-binding probe or probe set, one or more splints, and a plurality of oligonucleotides, wherein: (i) the target-binding probe or probe set comprises hybridization regions HR1, HR2, and HR3; (ii) the target nucleic acid comprises a hybridization region HR2′ that hybridizes to HR2; and (iii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints; and (b) ligating HR1 to a first adjacent oligonucleotide of the plurality of oligonucleotides, adjacent oligonucleotides of the plurality of oligonucleotides to one another, and HR3 to a second adjacent oligonucleotide of the plurality of oligonucleotides using the one or more splints as templates, thereby forming a circular probe that is hybridized to the target nucleic acid.

In some embodiments, the target nucleic acid can be contacted with the target-binding probe or probe set, one or more splints, and plurality of oligonucleotides simultaneously or sequentially.

In any of the provided embodiments, the target-binding probe or probe set can comprise a first probe that comprises hybridization regions HR1, HR2, and HR3.

In any of the provided embodiments, the first probe can comprise HR1, HR2, and HR3 in order from 5′ to 3′ or in order from 3′ to 5′.

In any of the provided embodiments, HR2′ can be a split hybridization region comprising adjacent hybridization regions HR2a′ and HR2b′; HR2 can comprise hybridization regions HR2a and HR2b that hybridize to HR2a′ and HR2b′, respectively; the target-binding probe or probe set can comprise (i) a first probe that comprises HR1 and HR2a; and (ii) a second probe that comprises HR2b and HR3; and HR2a can be ligated to HR2b using the target nucleic acid as a template.

In any of the provided embodiments, the ligating of HR2a to HR2b can comprise gap-filling using the target nucleic acid as a template, and the circular probe can comprise a gap-filled sequence. In some embodiments, the method can further comprise sequencing the gap-filled sequence or an amplification product of the circular probe comprising the gap-filled sequence.

In any of the provided embodiments, the one or more splints can comprise between or between about 1 and 10 splints.

In any of the provided embodiments, at least one of the one or more splints can hybridize to the ends of at least two oligonucleotides, wherein upon hybridization, a sequence between the ends of each of the at least two oligonucleotides can form a loop, and the sequence of each loop may not hybridize to the one or more splints. In any of the provided embodiments, each of the one or more splints can hybridize to the ends of at least two oligonucleotides, wherein upon hybridization, a sequence between the ends of each of the at least two oligonucleotides can form a loop, and the sequence of each loop may not hybridize to the one or more splints.

In any of the provided embodiments, the one or more splints can comprise at least two splints, and adjacent splints of the at least two splints can both hybridize to one of the plurality of oligonucleotides. In some embodiments, adjacent splints of the at least two splints can both hybridize to the ends of one of the plurality of oligonucleotides.

In any of the provided embodiments, the target nucleic acid can be in a sample, and the contacting, hybridizing, and ligating can be performed in situ.

In any of the provided embodiments, the target nucleic acid can comprise RNA. In any of the provided embodiments, the target nucleic acid can be an mRNA. In any of the provided embodiments, the target-binding probe or probe set can comprise DNA. In any of the provided embodiments, the first probe, one or more splints, and/or plurality of oligonucleotides can comprise DNA. In any of the provided embodiments, the first probe, one or more splints, and plurality of oligonucleotides can comprise DNA. In any of the provided embodiments, the first probe, second probe, one or more splints, and/or plurality of oligonucleotides can comprise DNA. In any of the provided embodiments, the first probe, second probe, one or more splints, and plurality of oligonucleotides can comprise DNA. In any of the provided embodiments, the one or more splints can comprise DNA. In any of the provided embodiments, the plurality of oligonucleotides can comprise DNA.

In some aspects, provided herein is a method for generating a circular probe, comprising: (a) contacting a sample comprising a first nucleic acid strand and a second nucleic acid strand in proximity to one another with a first probe, a second probe, and a plurality of oligonucleotides, wherein: (i) the first probe hybridizes to the first nucleic acid strand; (ii) the second probe hybridizes to the second nucleic acid strand; (iii) the first and second probes each comprise a plurality of hybridization regions that each hybridize to the ends of one or more of the plurality of oligonucleotides; wherein upon hybridization of two regions of a probe of the first and second probes to complementary sequences at the ends of an oligonucleotide of the plurality of oligonucleotides, a sequence of the oligonucleotide between the ends forms a loop, and the sequence of the loop does not hybridize to the first or second probe; and (iv) the first and second probes both hybridize to one or more of the plurality of oligonucleotides; and (b) ligating adjacent oligonucleotides of the plurality of oligonucleotides to one another using the first and second probes as templates, thereby forming a circular probe that is hybridized to the first and second probes.

In some embodiments, the sample can be contacted with the first probe, second probe, and plurality of oligonucleotides simultaneously or sequentially.

In any of the provided embodiments, the first nucleic acid strand and the second nucleic acid strand can be in the same molecule or in different molecules.

In any of the provided embodiments, one or both of the first and second probes can hybridize to the ends of at least two oligonucleotides, wherein upon hybridization, a sequence between the ends of each of the at least two oligonucleotides can form a loop, and the sequence of each loop may not hybridize to the first or second probe. In any of the provided embodiments, the first and second probes can each hybridize to the ends of at least two oligonucleotides, wherein upon hybridization, a sequence between the ends of each of the at least two oligonucleotides can form a loop, and the sequence of each loop may not hybridize to the first or second probe.

In any of the provided embodiments, the first and/or second nucleic acid strand can comprise RNA. In any of the provided embodiments, the first and second nucleic acid strand can comprise RNA. In any of the provided embodiments, the first and/or second nucleic acid strand can be an mRNA. In any of the provided embodiments, the first and second nucleic acid strand can be an mRNA. In any of the provided embodiments, the first probe, second probe, and/or plurality of oligonucleotides can comprise DNA. In any of the provided embodiments, the first probe, second probe, and plurality of oligonucleotides can comprise DNA.

In any of the provided embodiments, the plurality of oligonucleotides can comprise between or between about 2 and 20 oligonucleotides.

In any of the provided embodiments, the loop of at least 1, 2, 3, 4, 5, 6, 7, 8, 9, 10, 11, 12, 13, 14, 15, 16, 17, 18, 19, or 20 of the plurality of oligonucleotides can comprise a barcode sequence. In any of the provided embodiments, each loop of the plurality of oligonucleotides can comprise a barcode sequence. In any of the provided embodiments, the barcode sequences can be the same or different across the plurality of oligonucleotides.

In any of the provided embodiments, the method can further comprise detecting a sequence in the circular probe.

In any of the provided embodiments, the method can further comprise forming an amplification product using the circular probe as a template. In any of the provided embodiments, the method can further comprise forming an amplification product using the circular probe as a template, wherein one of the one or more splints can be used as a primer for forming the amplification product. In any of the provided embodiments, the method can further comprise forming an amplification product using the circular probe as a template, wherein the first probe or the second probe can be used as a primer for forming the amplification product.

In any of the provided embodiments, the method can further comprise detecting a sequence in the amplification product.

In any of the provided embodiments, the circular probe can comprise one or more cleavage sites, and the method can further comprise cleaving the one or more cleavage sites to form a subsequent probe. In some embodiments, the subsequent probe can comprise the plurality of oligonucleotides. In some embodiments, the subsequent probe may not comprise one or more of the plurality of oligonucleotides. In some embodiments, the one or more of the plurality of oligonucleotides can be removed by the cleaving.

In any of the provided embodiments, the cleaving can be subsequent to the detecting.

In any of the provided embodiments, the method can further comprise (a) contacting the subsequent probe with one or more additional splints and one or more additional oligonucleotides, wherein: the one or more additional splints can comprise a plurality of hybridization regions that each hybridize to the ends of at least one of the one or more additional oligonucleotides, wherein a sequence between the ends can form a loop upon additional oligonucleotide-additional splint hybridization, and the loop may not hybridize to the one or more additional splints; and the one or more additional splints can hybridize to the ends of the subsequent probe; and (b) ligating each end of the subsequent probe to an adjacent additional oligonucleotide of the one or more additional oligonucleotides using the one or more additional splints as templates and optionally ligating adjacent additional oligonucleotides to each other using the one or more additional splints as templates, thereby forming a subsequent circular probe. In some embodiments, adjacent additional oligonucleotides can be ligated to each other using the one or more additional splints as templates.

In any of the provided embodiments, the one or more additional splints can comprise between or between about 1 and 10 splints.

In any of the provided embodiments, the one or more additional oligonucleotides can comprise between or between about 1 and 20 oligonucleotides.

In any of the provided embodiments, the loop of at least 1, 2, 3, 4, 5, 6, 7, 8, 9, 10, 11, 12, 13, 14, 15, 16, 17, 18, 19, or 20 of the one or more additional oligonucleotides can comprise a barcode sequence. In any of the provided embodiments, each loop of the one or more additional oligonucleotides can comprise a barcode sequence.

In any of the provided embodiments, the method can further comprise detecting a sequence of the subsequent circular probe or an amplification product of the subsequent circular probe.

In any of the provided embodiments, one or more of the contacting, ligating, and detecting steps can be repeated.

In any of the provided embodiments, the detecting can comprise sequencing all or a portion of the circular probe or subsequent circular probe and/or in situ hybridization to the circular probe or subsequent circular probe. In any of the provided embodiments, the sequencing can comprise sequencing hybridization, sequencing by ligation, and/or fluorescent in situ sequencing. In any of the provided embodiments, the in situ hybridization can comprise sequential fluorescent in situ hybridization. In any of the provided embodiments, the detecting can comprise hybridizing to the circular probe or subsequent circular probe a detection oligonucleotide labeled with a fluorophore, an isotope, a mass tag, or a combination thereof.

In any of the provided embodiments, the detecting can comprise imaging the circular probe or subsequent circular probe. In any of the provided embodiments, the target nucleic acid can be in a sample, and the detecting can be performed in situ.

In any of the provided embodiments, the sample can be a fixed and/or permeabilized biological sample. In any of the provided embodiments, the sample can be a tissue sample. In any of the provided embodiments, the sample can be a formalin-fixed, paraffin-embedded (FFPE) tissue sample, a frozen tissue sample, or a fresh tissue sample. In any of the provided embodiments, the sample can be a tissue slice between about 1 μm and about 50 μm in thickness, optionally wherein the tissue slice is between about 5 μm and about 35 μm in thickness. In any of the provided embodiments, the tissue slice is between about 5 μm and about 35 μm in thickness. In any of the provided embodiments, the sample can be crosslinked. In any of the provided embodiments, the sample can be embedded in a hydrogel matrix. In any of the provided embodiments, the sample may not embedded in a hydrogel matrix. In any of the provided embodiments, the sample can be cleared.

In some aspects, provided herein is a kit for generating a circular probe, comprising: a first probe, one or more splints, and a plurality of oligonucleotides, wherein: (i) the first probe comprises hybridization regions HR1, HR2, and HR3, wherein HR2 hybridizes to hybridization region HR2′ of a target nucleic acid; and (ii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints.

In some aspects, provided herein is a kit for generating a circular probe, comprising: a first probe, a second probe, one or more splints, and a plurality of oligonucleotides, wherein: (i) the first probe comprises hybridization regions HR1 and HR2a; (ii) the second probe comprises hybridization regions HR2b and HR3; wherein HR2a and HR2b hybridize to adjacent hybridization regions HR2a′ and HR2b′, respectively, of a target nucleic acid; and (iii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints.

In some aspects, provided herein is a kit for generating a circular probe, comprising: a first probe, a second probe, and a plurality of oligonucleotides, wherein: (i) the first probe hybridizes to a first nucleic acid strand in a sample; (ii) the second probe hybridizes to a second nucleic acid strand in the sample; wherein the first and second nucleic acid strands are in proximity to one another; (iii) the first and second probes each comprise a plurality of hybridization regions that each hybridize to the ends of one or more of the plurality of oligonucleotides; wherein upon hybridization of two regions of a probe of the first and second probes to complementary sequences at the ends of an oligonucleotide of the plurality of oligonucleotides, a sequence of the oligonucleotide between the ends forms a loop, and the sequence of the loop does not hybridize to the first or second probe; and (iv) the first and second probes both hybridize to one or more of the plurality of oligonucleotides.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 and FIG. 4 depict an exemplary method for generating a circular probe that is hybridized to a target nucleic acid of interest. The circular probe can be formed using a first probe (e.g., a U-probe) that hybridizes to the target nucleic acid, one or more splints that hybridize to the ends of the first probe, and a plurality of oligonucleotides whose ends hybridize to the one or more splints. The plurality of oligonucleotides can each include a loop sequence between the hybridizing ends that does not hybridize to other sequences. The loop sequences can include barcode sequences. The barcode sequences can be the same or different across loop sequences. The first probe (e.g., a U-probe) and plurality of oligonucleotides can be ligated to one another to form a circular probe. The one or more splints can be used as templates for the ligation. The circular probe can be optionally amplified prior to steps of detecting sequences therein or in an amplification product thereof. In some embodiments, one of the splints can be used as a primer for the amplification (FIG. 1).

FIG. 2 and FIG. 5 depict an exemplary method for generating a circular probe that is hybridized to a target nucleic acid of interest. The circular probe can be formed using a first probe and a second probe that hybridize to the target nucleic acid, one or more splints that hybridize to the ends of the first probe and second probe, and a plurality of oligonucleotides whose ends hybridize to the one or more splints. The plurality of oligonucleotides can each include a loop sequence between the hybridizing ends that does not hybridize to other sequences. The loop sequences can include barcode sequences. The barcode sequences can be the same or different across loop sequences. The first probe, second probe, and plurality of oligonucleotides can be ligated to one another to form a circular probe. The one or more splints can be used as templates for the ligation of the first probe and the second probe to the plurality of oligonucleotides and for the ligation of the plurality of oligonucleotides to one another. The target nucleic acid can be used as a template for the ligation of the first probe and the second probe to one another. The circular probe can be optionally amplified prior to steps of detecting sequences therein or in an amplification product thereof. In some embodiments, one of the splints can be used as a primer for the amplification (FIG. 5).

FIG. 3 depicts an exemplary method for generating a circular probe that can be used, for example, to detect two nucleic acids in proximity with one another. The method can allow for spatial analysis of nucleic acid strands contained in a sample. The circular probe can be formed using a first probe that hybridizes to a first nucleic acid strand, a second probe that hybridizes to a second nucleic acid strand, and a plurality of oligonucleotides whose ends hybridize to the first and second probes. The plurality of oligonucleotides can each include a loop sequence between the hybridizing ends that does not hybridize to other sequences. The loop sequences can include barcode sequences. The barcode sequences can be the same or different across loop sequences. The plurality of oligonucleotides can be ligated to one another to form a circular probe. The first and second probes can be used as templates (e.g., as splints) for the ligation. The circular probe can be optionally amplified prior to steps of detecting sequences therein or in an amplification product thereof.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods for generating large circular probes, for instance circular probes containing multiple barcode sequences. In some embodiments, the circular probes are formed from a plurality of oligonucleotides containing loop sequences that, e.g., do not hybridize to other polynucleotides. In some embodiments, the loop sequences contain the barcode sequences. In some embodiments, portions of the oligonucleotides hybridize to primary probes (e.g., a U probe or first probe) or splints targeting a nucleic acid sequence or region of interest. In some embodiments, the number of oligonucleotides is greater than the number of splints and/or probes to which they hybridize. In some embodiments, the probes, splints, or target nucleic acid sequence are used as templates for ligation of the oligonucleotides to form the circular probe. In some embodiments, the circular probe is amplified to form an amplification product. In some embodiments, a sequence is detected in the circular probe or amplification product thereof. Also provided herein are compositions and kits for performing the provided methods.

In some embodiments, the target nucleic acid is in a sample. In some embodiments, the provided methods involve contacting nucleic acid strands, e.g., a first and second nucleic acid strand, with the oligonucleotides, wherein the nucleic acid strands are in a sample. In some embodiments, the sample is a biological sample. In some embodiments, the circular probe or amplification product thereof is analyzed in situ.

A drawback to traditional padlock or circular probes is an upper bound length limitation on the linear oligonucleotides that are circularized to form the circular probe. Limits on the length of such oligonucleotides are both functional and practical. Functionally, the longer an oligonucleotide sequence is, the more likely a sequence error is to result due to problems with the synthesis reaction. Errors in synthesis can lead to linear probes that are unable to circularize and thus are not able to serve as templates for rolling circle amplification. For example, assuming a linear oligonucleotide has a length of 100 bases, a synthesis error that occurs downstream of the initial sequence may produce a molecule that can hybridize to a target nucleic acid but is unable to circularize. In some examples, the synthesis error may result in a linear oligonucleotide of which the 5′ end can hybridize to a target nucleic acid (e.g., due to the 5′ end being synthesized first and thus being more accurate than the 3′ end), but the 3′ end contains a truncation or base error that abolishes templated ligation. In these examples, the erroneous padlock probes occupy the target nucleic acid but do not yield a successfully ligated circular probe for rolling circle amplification, leading to less efficient detection. Practically, synthesizing oligonucleotides significantly greater than 100 bases is typically not cost effective.

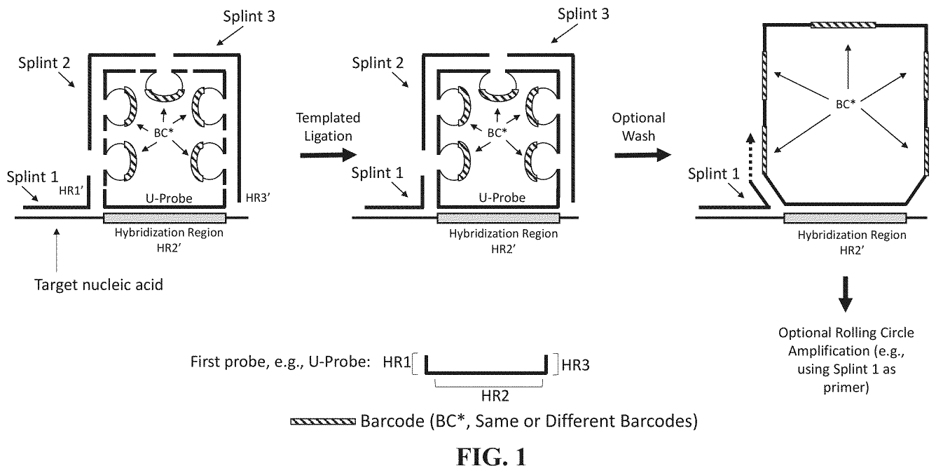

In some embodiments, the polynucleotides described here address the length limitations of traditional padlock or circular probes because multiple polynucleotides can be assembled into large circular probes, thus overcoming the bottleneck of oligonucleotide synthesis and making it possible to increase the barcoding space/potential within the circular probes. In some embodiments, one or more primary probes are hybridized to target nucleic acids. In some embodiments, one or more splints are hybridized to the primary probes. In some embodiments, a plurality of oligonucleotides can then be hybridized to the primary probes or the one or more splints such that, following ligation, a large circular probe can be formed that incorporates the oligonucleotides and optionally the primary probes. In some aspects, the barcode sequences are contained in loop sequences of the oligonucleotides that do not hybridize to other polynucleotides (see, e.g., FIG. 1-5). Thus, in some aspects the sequence length of the circular probe is greater than the combined length of the hybridization regions of the polynucleotides (e.g., of the oligonucleotides and optionally primary probes) forming the circular probe. In this manner, a larger circular probe can be generated in which more sequence space can be devoted to barcode sequences. In some aspects, the multiple barcode sequences incorporated may allow increased signal intensity during detection of the barcode sequences. In some aspects, the multiple barcode sequences incorporated may allow an increase in encoding space by allowing use of various combinations of the barcodes. In some embodiments, the ligation of the plurality of oligonucleotides to the primary probe to generate a large circular probe is performed in situ.

Nucleic acids and/or analytes that can be analyzed by the presently disclosed methods are described in greater detail in Section II.

### II. Samples, Analytes, and Target Sequences

A. Samples

A sample disclosed herein can be derived from any biological sample. Methods, probes, and kits disclosed herein may be used for analyzing a biological sample, which may be obtained from a subject using any of a variety of techniques including, but not limited to, biopsy, surgery, and laser capture microscopy (LCM), and generally comprises cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a predisposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe or probe set (e.g., probes, splints, and oligonucleotides described herein). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., probes, splints, and oligonucleotides described herein).

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel comprises a hybrid material, e.g., the hydrogel material comprises elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate comprises a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, probes that enter the sample and bind to analytes therein may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, TritonX100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used herein include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, probes that are specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods, probes, and kits disclosed herein can be used to detect and analyze a wide variety of different analytes, for instance target nucleic acids or nucleic acid strands. In some aspects, an analyte can include any biological substance, structure, moiety, or component to be analyzed. In some aspects, a target disclosed herein may similarly include any analyte of interest. In some examples, a target or analyte can be directly or indirectly detected. In some embodiments, the target nucleic acid is any of the nucleic acid analytes described herein. In some embodiments, the nucleic acid strands are any of the nucleic acid analytes described herein.

Analytes can be derived from a specific type of cell and/or a specific subcellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule, macromolecule, or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g., a circularizable probe or probe set). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules (e.g., cellular nucleic acids), such as DNA (e.g., genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins or prions or any molecule which comprises a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g., interactions between proteins and nucleic acids, e.g., regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods, probes, and kits disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprises one or more single-stranded target sequences.

(ii) Labelling Agents

In some embodiments, provided herein are methods, probes, and kits for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode comprises a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety comprises one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes comprises a single species of analyte (e.g., a single species of polypeptide). In some embodiments in which the plurality of analytes comprises a single species of analyte, the analyte binding moieties of the plurality of analyte labelling agents are the same. In some embodiments in which the plurality of analytes comprises a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes comprises multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods, probes, and kits for analyzing one or more products of an endogenous analyte and/or a labelling agent in a biological sample. In some embodiments, a circular probe (e.g., formed using the probes, splints, and oligonucleotides described in Section III) can be used for analyzing an endogenous analyte, one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or an exogenous molecule such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. In some instances, various probes and probe sets can be used to generate a product comprising a target sequence that can be hybridized by a nucleic acid complex described herein (e.g., formed using the probes, splints, and oligonucleotides described in Section III). In some instances, a probe or probe set disclosed herein is a circularizable probe or probe set comprising a barcode region comprising one or more barcode sequences. Exemplary barcoded probes or probe sets may be based on a circularizable probe or probe set (e.g., a padlock probe or a gapped padlock probe), a SNAIL (Splint Nucleotide Assisted Intramolecular Ligation) probe set, a PLAYR (Proximity Ligation Assay for RNA) probe set, a PUSH (Proximity Ligation in situ hybridization) probe set, and RNA-templated ligation probes. The specific probe or probe set design can vary.

(b) Ligation

In some embodiments, a product of an endogenous analyte and/or a labelling agent is a ligation product that may comprise a target sequence that can be hybridized by the nucleic acid complexes described herein (e.g., formed using the probes, splints, and oligonucleotides described in Section III). In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent or probe, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety.

In some embodiments, a circular or circularizable probe or probe set (e.g., formed using the probes, splints, and oligonucleotides described in Section III) may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using circular or circularizable probes and rolling circle amplification of circular or circularized probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set (e.g., probes, splints, and oligonucleotides that can form a circular probe). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe (e.g., formed using the probes, splints, and oligonucleotides described in Section III). In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe or probe (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

C. Target Sequences

A target sequence for a probe disclosed herein (e.g., bound directly or indirectly by a detectably labelled probe and/or a non-detectably labelled probe) may be comprised in any analyte disclose herein, including an endogenous analyte (e.g., a viral or cellular nucleic acid), a labelling agent, or a product or derivative of an endogenous analyte and/or a labelling agent. For example, a target sequence for detection is comprised by a product generated by hybridizing a probe or probe set (e.g., comprising the probes, splints, and oligonucleotides as described in Section III) to an endogenous analyte and performing RCA using the probe or probe set.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594, US 2021/0164039, U.S. Pat. No. 11,008,608, and US 2021/0238665, all of which are hereby incorporated by reference in their entireties.

### III. Probes, Splints, and Oligonucleotides for Generating Circular Probes

Disclosed herein in some aspects are nucleic acid probes, splints, and oligonucleotides that are introduced into a cell or used to otherwise contact a biological sample such as a tissue sample. The nucleic acid probes, splints, and oligonucleotides may comprise any of a variety of entities that can hybridize to a nucleic acid, typically by Watson-Crick base pairing, such as DNA, RNA, LNA, PNA, etc. The nucleic acid probes typically contain a targeting sequence that is able to directly or indirectly bind to at least a portion of a target nucleic acid. The nucleic acid probes may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

Provided herein are methods involving the use of one or more probes for analyzing one or more target nucleic acid(s), such as a target nucleic acid (for example, a messenger RNA) present in a cell or a biological sample, such as a tissue sample. Also provided are probes, sets of probes, compositions, kits, systems, and devices for use in accordance with the provided methods. In some aspects, the provided methods and systems can be applied to detect, image, quantitate, or determine the presence or absence of one or more target nucleic acid(s) or portions thereof (e.g., presence or absence of sequence variants such as point mutations and SNPs). In some aspects, the provided methods can be applied to detect, image, quantitate, or determine the sequence of one or more target nucleic acid(s), comprising sequence variants such as point mutations and SNPs.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, the provided methods involve a step of contacting, or hybridizing, one or more polynucleotides, such as any of the probes, splints, and/or oligonucleotides described herein, to a cell or a sample containing a target nucleic acid with a region of interest in order to form a hybridization complex. In some aspects, the provided methods comprise one or more steps of ligating the polynucleotides, for instance of ligating the probes and/or oligonucleotides to form a circular probe. In some aspects, the provided methods involve a step of amplifying one of the polynucleotides (e.g., a circular probe), to generate an amplification product. In some aspects, the provided methods involve a step of detecting and/or determining the sequence of all or a portion of the amplification product (for example, of one or more barcodes contained in the amplification product) and/or one or more of the polynucleotides, for instance the circular probe, with or without amplification, for instance any barcodes contained therein. In some aspects, the provided methods involve performing one or more of the steps described herein, simultaneously and/or sequentially.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the probes, splints, and/or oligonucleotides comprises DNA. In some embodiments, the target nucleic acid is RNA, and the probes, splints, and/or oligonucleotides comprise DNA.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest.

In some aspects, the methods disclosed herein involve the use of one or more probes or probe sets that hybridize to a target nucleic acid, such as an RNA molecule.

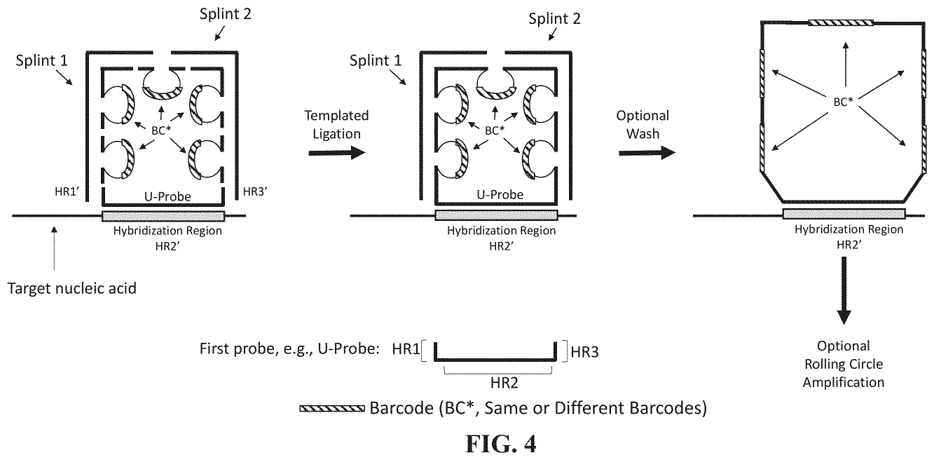

In some embodiments, the target nucleic acid includes a hybridization region HR2′ to which the one or more probes or probe sets hybridize. In some embodiments, the target nucleic acid is contacted with a first probe that includes a hybridization region HR2 that hybridizes to HR2′ (see, e.g., FIG. 1 and FIG. 4). In some embodiments, the first probe further includes hybridization regions HR1 and HR3. In some embodiments, HR1 and HR3 do not hybridize to the target nucleic acid. In some embodiments, HR2 is between HR1 and HR3. In some aspects, the first probe is a U-shaped probe as depicted in FIG. 1 and FIG. 4. In some embodiments, the first probe includes, from 5′ to 3′, hybridization regions HR1, HR2, and HR3. In some embodiments, the first probe includes, from 3′ to 5′, hybridization regions HR1, HR2, and HR3. In some embodiments, the ends of the first probe do not hybridize to the target nucleic acid. In some embodiments, HR1 is at one of the ends of the first probe, and HR3 is at the other end of the first probe.

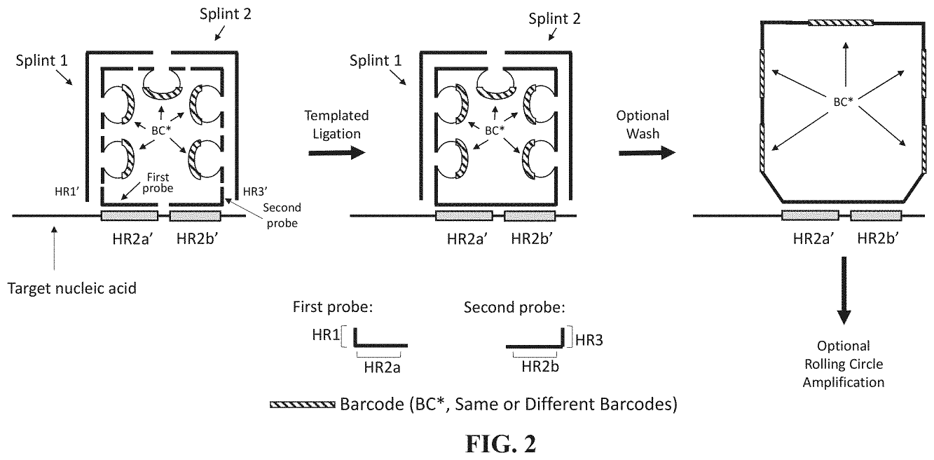

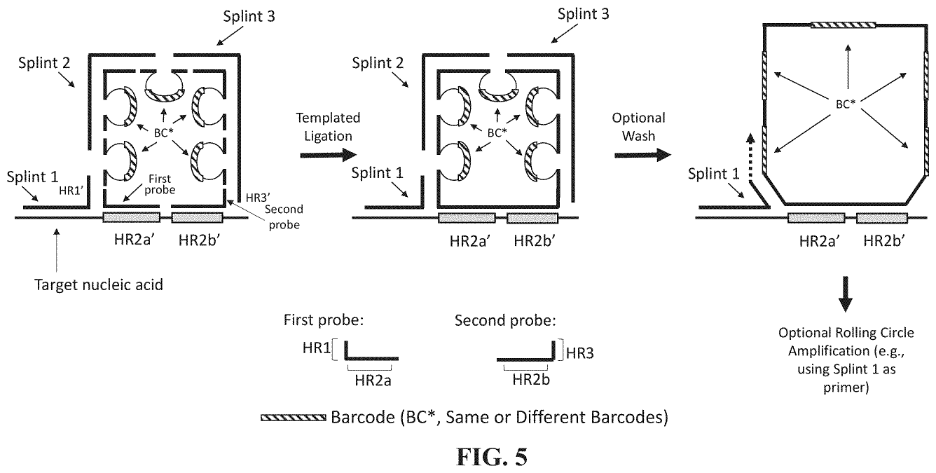

In some embodiments, the target nucleic acid includes hybridization regions HR2a′ and HR2b′. In some embodiments, HR2a′ and HR2b′ are adjacent to one another. In some embodiments, the target nucleic acid is contacted with a first probe and a second probe that hybridize to HR2a′ and HR2b′, respectively (see, e.g., FIG. 2 and FIG. 5). In some embodiments, the first probe includes hybridization region HR2a that hybridizes to HR2a′. In some embodiments, the first probe also includes hybridization region HR1. In some embodiments, HR1 does not hybridize to the target nucleic acid. In some embodiments, the second probe includes hybridization region HR2b that hybridizes to HR2b′. In some embodiments, the second probe also includes hybridization region HR3. In some embodiments, HR3 does not hybridize to the target nucleic acid. In some embodiments, HR1 is at one of the ends of the first probe, and HR2a is at the other end of the first probe. In some embodiments, HR3 is at one of the ends of the second probe, and HR2b is at the other end of the first probe.

In some embodiments, the target nucleic acid includes hybridization regions HR2a′ and HR2b′ and additional HR2′ hybridization regions that can be hybridized to a first probe and a second probe and additional probes, respectively. In some embodiments, any of the first probe, second probe, or additional probes includes a hybridization region that hybridizes to HR2a′, HR2b′, and additional HR2′ hybridization regions and an overhang region on each of the 3′ end and 5′ end that does not hybridize to the target nucleic acid. In some embodiments, any of the overhang regions of the first probe, second probe and additional probes may be used for hybridizing to a splint. In some embodiments, a 3′ end and a 5′ end of the first probe, second probe, or additional probes can be ligated to each other using the one or more splints as a template. In some instances, the generated circular probe comprises 3 or more hybridization regions that hybridize to 3 or more separate probes. In some embodiments, the methods disclosed herein involve the use of one or more probes that each hybridize to the target nucleic acid. In some embodiments, the one or more probes include between or between about 1 and 10 splints, inclusive, for instance 1, 2, 3, 4, 5, 6, 7, 8, 9, or 10 probes.

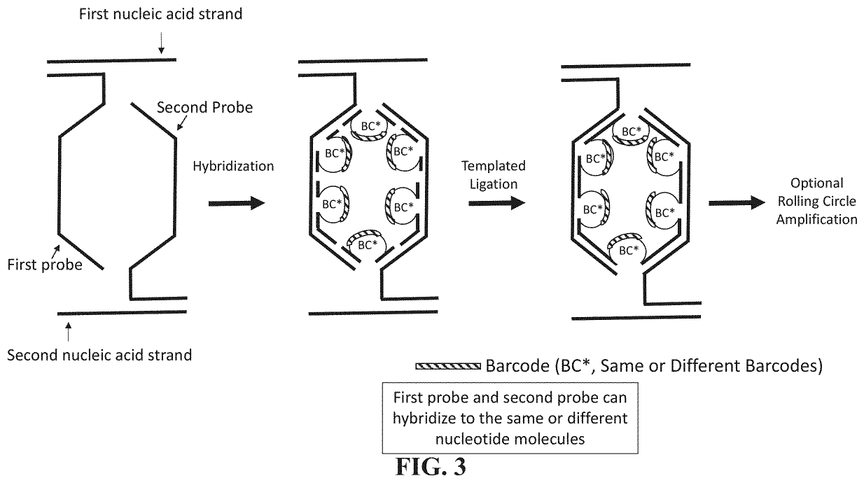

In some embodiments, the target nucleic acid is contacted with a first probe and a second probe that hybridize to a first nucleic acid strand and a second nucleic acid strand, respectively. In some embodiments, the first and second nucleic acid strands are in proximity to one another (see, e.g., FIG. 3). In some embodiments, the first and second nucleic acid strands are in the same molecule. In some embodiments, the first and second nucleic acid strands are in different molecules.

In some embodiments, the methods disclosed herein further involve the use of one or more splints that hybridize to the one or more probes or probe sets and/or the target nucleic acid (see, e.g., FIGS. 1, 2, 4, and 5). In some embodiments, the one or more splints include at least 1, 2, 3, 4, 5, 6, 7, 8, 9, or 10 splints. In some embodiments, the one or more splints include up to 10 splints. In some embodiments, the one or more splints include between or between about 1 and 10 splints, 1 and 9 splints, 1 and 8 splints, 1 and 7 splints, 1 and 6 splints, 1 and 5 splints, 1 and 4 splints, 1 and 3 splints, 2 and 10 splints, 2 and 9 splints, 2 and 8 splints, 2 and 7 splints, 2 and 6 splints, 2 and 5 splints, 2 and 4 splints, 3 and 10 splints, 3 and 9 splints, 3 and 8 splints, 3 and 7 splints, 3 and 6 splints, 3 and 5 splints, 4 and 10 splints, 4 and 9 splints, 4 and 8 splints, 4 and 7 splints, 4 and 6 splints, 5 and 10 splints, 5 and 9 splints, 5 and 8 splints, 5 and 7 splints, 6 and 10 splints, 6 and 9 splints, 6 and 8 splints, 7 and 10 splints, 7 and 9 splints, and 8 and 10 splints, each inclusive. In some embodiments, the one or more splints include a hybridization region HR1′ that hybridizes to HR1. In some embodiments, the one or more splints also include a hybridization region HR3′ that hybridizes to HR3. In some embodiments, the one or more splints also hybridize to the target nucleic acid (see, e.g., FIG. 1 and FIG. 5). In some embodiments, the one or more splints do not hybridize to the target nucleic acid (see, e.g., FIG. 2 and FIG. 4).

In some embodiments, the methods disclosed herein further involve the use of a plurality of oligonucleotides that hybridize to the one or more probes, probe sets, and/or one or more splints (see, e.g., FIG. 1-5). In some embodiments, the plurality of oligonucleotides includes at least 2, 3, 4, 5, 6, 7, 8, 9, 10, 11, 12, 13, 14, 15, 16, 17, 18, 19, or 20 oligonucleotides. In some embodiments, the plurality of oligonucleotides includes up to 20 oligonucleotides. In some embodiments, the plurality of oligonucleotides include between or between about 2 and 20 oligonucleotides, 2 and 18 oligonucleotides, 2 and 16 oligonucleotides, 2 and 14 oligonucleotides, 2 and 12 oligonucleotides, 2 and 10 oligonucleotides, 2 and 8 oligonucleotides, 2 and 6 oligonucleotides, 2 and 4 oligonucleotides, 4 and 20 oligonucleotides, 4 and 18 oligonucleotides, 4 and 16 oligonucleotides, 4 and 14 oligonucleotides, 4 and 12 oligonucleotides, 4 and 10 oligonucleotides, 4 and 8 oligonucleotides, 4 and 6 oligonucleotides, 6 and 20 oligonucleotides, 6 and 18 oligonucleotides, 6 and 16 oligonucleotides, 6 and 14 oligonucleotides, 6 and 12 oligonucleotides, 6 and 10 oligonucleotides, 6 and 8 oligonucleotides, 8 and 20 oligonucleotides, 8 and 18 oligonucleotides, 8 and 16 oligonucleotides, 8 and 14 oligonucleotides, 8 and 12 oligonucleotides, 8 and 10 oligonucleotides, 10 and 20 oligonucleotides, 10 and 18 oligonucleotides, 10 and 16 oligonucleotides, 10 and 14 oligonucleotides, 10 and 12 oligonucleotides, 12 and 20 oligonucleotides, 12 and 18 oligonucleotides, 12 and 16 oligonucleotides, 12 and 14 oligonucleotides, 14 and 20 oligonucleotides, 14 and 18 oligonucleotides, 14 and 16 oligonucleotides, 16 and 20 oligonucleotides, 16 and 18 oligonucleotides, or 18 and 20 oligonucleotides, each inclusive. In some embodiments, the plurality of oligonucleotides include between or between about 2 and 20 oligonucleotides, for instance 2, 3, 4, 5, 6, 7, 8, 9, 10, 11, 12, 13, 14, 15, 16, 17, 18, 19, or 20 oligonucleotides. In some embodiments, the number of oligonucleotides is greater than the number of splints or probes, for instance is 2, 3, 4, 5, 6, 7, 8, 9, or 10 times the number of splints or probes.

In some embodiments, the one or more probes and/or the one or more splints include a plurality of hybridization regions that each hybridize to one or more of the plurality of oligonucleotides. In some embodiments, each of the plurality of hybridization regions hybridizes to the ends of one or more of the plurality of oligonucleotides. In some embodiments, hybridization regions of the splints for hybridizing to the ends of multiple oligonucleotides may comprise a common sequence. In some aspects, a splint may comprise repeating sequences for hybridizing to the oligonucleotides. In some embodiments, hybridization regions of the splint for hybridizing to the probe (e.g., first and/or second probe) is a sequence specific for the probe. In some embodiments, upon hybridization of two regions of a splint or probe to complementary sequences at the ends of an oligonucleotide, a sequence of the oligonucleotide between the ends forms a loop. In some embodiments, the sequence of the loop does not hybridize to the one or more splints and/or to the one or more probes (see, e.g., FIG. 1-5). In some embodiments, upon hybridization of two regions of one splint to the two ends of the oligonucleotide, the non-hybridizing internal sequence of the oligonucleotide is positioned into the loop configuration.

In some embodiments, the combined length of the first probe and the plurality of oligonucleotides is greater than the combined length of HR2′ and the regions of the one or more splints that hybridize to the plurality of oligonucleotides (see, e.g., FIG. 1 and FIG. 4). In some embodiments, the combined length of the first probe, the second probe, and the plurality of oligonucleotides is greater than the combined length of HR2a′, HR2b′, and the regions of the one or more splints that hybridize to the plurality of oligonucleotides (see, e.g., FIG. 2 and FIG. 5). In some embodiments, the combined length of the plurality of oligonucleotides is greater than the combined length of the regions of the first and second probes that hybridize to the plurality of oligonucleotides (see, e.g., FIG. 3). In some aspects, this increase is due at least in part to the inclusion of a loop sequence in one or more oligonucleotides that does not hybridize to the one or more splints, target nucleic acid, or one or more probes.

In some embodiments, at least 1, 2, 3, 4, 5, 6, 7, 8, 9, or 10 of the one or more splints hybridizes to at least two oligonucleotides. In some embodiments, each of the one or more splints hybridizes to at least two oligonucleotides. In some embodiments, at least 1, 2, 3, 4, 5, 6, 7, 8, 9, or 10 of the one or more splints hybridizes to at least three oligonucleotides. In some embodiments, each of the one or more splints hybridizes to at least three oligonucleotides. In some embodiments, at least 1, 2, 3, 4, 5, 6, 7, 8, 9, or 10 of the one or more splints hybridizes to at least four oligonucleotides. In some embodiments, each of the one or more splints hybridizes to at least four oligonucleotides. In some embodiments, at least 1, 2, 3, 4, 5, 6, 7, 8, 9, or 10 of the one or more splints hybridizes to at least five oligonucleotides. In some embodiments, each of the one or more splints hybridizes to at least five oligonucleotides.

In some embodiments, at least one of the one or more probes hybridizes to at least two oligonucleotides. In some embodiments, each of the one or more probes hybridizes to at least two oligonucleotides. In some embodiments, at least one of the one or more probes hybridizes to at least three oligonucleotides. In some embodiments, each of the one or more probes hybridizes to at least three oligonucleotides. In some embodiments, at least one of the one or more probes hybridizes to at least four oligonucleotides. In some embodiments, each of the one or more probes hybridizes to at least four oligonucleotides. In some embodiments, at least one of the one or more probes hybridizes to at least five oligonucleotides. In some embodiments, each of the one or more probes hybridizes to at least five oligonucleotides.

In some embodiments, the one or more splints include at least two splints, and adjacent splints both hybridize to one of the plurality of oligonucleotides (e.g., both hybridize to the same oligonucleotide). In some embodiments, adjacent splints both hybridize to the ends of one of the plurality of oligonucleotides (e.g., both hybridize to the ends of the same oligonucleotide; see, e.g., FIGS. 1, 2, 4, and 5).

In some embodiments, the first and second probes both hybridize to one or more of the plurality of oligonucleotides (e.g., both hybridize to the same oligonucleotide). In some embodiments, the first and second probes both hybridize two of the plurality of oligonucleotides (e.g., both hybridize to the same two oligonucleotides; see, e.g., FIG. 3).

In some embodiments, at least one of the plurality of oligonucleotides includes a barcode sequence. In some embodiments, at least 1, 2, 3, 4, 5, 6, 7, 8, 9, 10, 11, 12, 13, 14, 15, 16, 17, 18, 19, or 20 of the plurality of oligonucleotides includes a barcode sequence. In some embodiments, each of the plurality of oligonucleotides includes a barcode sequence. In some embodiments, the barcode sequence is contained within the loop sequence of the oligonucleotide. In some embodiments, the barcode sequences are the same across the plurality of oligonucleotides used to form one circular probe. In some embodiments, the plurality of oligonucleotides used to form one circular probe include at least 2, 3, 4, 5, 6, 7, 8, 9, 10, 11, 12, 13, 14, 15, 16, 17, 18, 19, or 20 different barcode sequences. In some embodiments, the barcode sequences or combination of barcode sequences used to form one circular probe is unique and associated with the corresponding target nucleic acid.

In some embodiments, at least one of the plurality of oligonucleotides includes more than one barcode sequence. If more than one barcode sequence is present in an oligonucleotide, the barcode sequences may both be in the loop sequence of the oligonucleotide. The barcode sequences may be positioned next to each other and/or interspersed with other sequences. In some embodiments, two or more of the barcode sequences may also at least partially overlap. In some embodiments, two or more of the barcode sequences in the same oligonucleotide do not overlap. In some embodiments, all of the barcode sequences in the same oligonucleotide are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the target nucleic acid and/or the sample containing the target nucleic acid is contacted with the one or more probes and the plurality of oligonucleotides simultaneously. In some embodiments, the target nucleic acid and/or the sample containing the target nucleic acid is contacted with the one or more probes and the plurality of oligonucleotides sequentially. In some embodiments, the target nucleic acid and/or the sample containing the target nucleic acid is contacted with the one or more probes before being contacted with the plurality of oligonucleotides. In some embodiments, the target nucleic acid and/or the sample containing the target nucleic acid is contacted with the one or more probes after being contacted with the plurality of oligonucleotides.

In some embodiments, the target nucleic acid and/or the sample containing the target nucleic acid is contacted with the one or more probes, the one or more splints, and the plurality of oligonucleotides simultaneously. In some cases, to detect multiple target nucleic acids, the sample can be contacted with different sets of probes, splints, and oligonucleotides for each target nucleic acid. In some embodiments, the target nucleic acid and/or the sample containing the target nucleic acid is contacted with the one or more probes, the one or more splints, and the plurality of oligonucleotides sequentially. In some embodiments, the target nucleic acid and/or the sample containing the target nucleic acid is contacted with the one or more probes before being contacted with the one or more splints and the plurality of oligonucleotides. In some embodiments, the target nucleic acid and/or the sample containing the target nucleic acid is contacted with the one or more probes and the one or more splints before being contacted with the plurality of oligonucleotides. In some embodiments, the target nucleic acid and/or the sample containing the target nucleic acid is contacted with the one or more probes and the one or more splints after being contacted with the plurality of oligonucleotides. IV. Ligation, Amplification, and Detection of Circular Probes

In some embodiments, the methods disclosed herein further involve ligating one or more probes and/or one or more oligonucleotides to one another in order to form a circular probe. Any suitable methods and conditions for hybridization of probes, ligation, amplification, and detection may be used, e.g., as described in Sections II-IV. In some embodiments, the circular probe includes sequences of the plurality of oligonucleotides and the one or more probes (see, e.g., FIGS. 1, 2, 4, and 5). In some embodiments, the circular probe includes sequences of the plurality of oligonucleotides (see, e.g., FIG. 3). In some embodiments, the circular probe is hybridized directly to the target nucleic acid (see, e.g., FIGS. 1, 2, 4, and 5). In some embodiments, the circular probe is hybridized to one or more probes that are hybridized to target nucleic acids (see, e.g., FIG. 3). In some embodiments, ligation and generation of the circular probe is dependent on the hybridizations between the target nucleic acid and probe(s) and the hybridizations between the splints and oligonucleotides/probe(s). In some embodiments, the methods disclosed herein further include forming an amplification product using the circular probe as a template. In some embodiments, a probe or a splint is used as a primer for forming the amplification product (see, e.g., FIG. 1 and FIG. 5).

In some embodiments, the circular probe is directly hybridized to the target nucleic acid. In some embodiments, the circular probe is formed from a probe or probe set (e.g., comprising the oligonucleotides described herein, and optionally the one or more probes described herein) that is capable of DNA-templated ligation. See, e.g., U.S. Pat. No. 8,551,710, which is hereby incorporated by reference in its entirety. In some embodiments, the circular probe is formed from a probe or probe set (e.g., comprising the oligonucleotides described herein, and optionally the one or more probes described herein) that is capable of RNA-templated ligation. In some embodiments, the probe that hybridizes to the target nucleic acid can be of any suitable design and modified to be ligated to the oligonucleotides by introduction of the splints described herein. Exemplary RNA-templated ligation probes and methods are described in US 2020/0224244, which is incorporated herein by reference in its entirety. In some embodiments, the circular probe is formed from a specific amplification of nucleic acids via intramolecular ligation (e.g., SNAIL) probe set. See, e.g., U.S. Pat. Pub. 2019/0055594, which is hereby incorporated by reference in its entirety. In some embodiments, the circular probe is formed from a probe capable of proximity ligation, for instance a proximity ligation assay for RNA (e.g., PLAYR) probe set. See, e.g., U.S. Pat. Pub. 2016/0108458, which is hereby incorporated by reference in its entirety.

In some embodiments, the circular probe is indirectly hybridized to the target nucleic acid. In some embodiments, the circular probe is formed from a probe set (e.g., comprising the oligonucleotides described herein, and optionally the one or more probes described herein) that is capable of proximity ligation, for instance a proximity ligation in situ hybridization (PLISH) probe set. See, e.g., U.S. Pat. Pub. 2020/0224243, which is hereby incorporated by reference in its entirety.

In some embodiments, a 3′ end and a 5′ end of the probes can be ligated using the target nucleic acid (e.g., RNA) as a template. In some embodiments, a 3′ end and a 5′ end of polynucleotides of the oligonucleotides and/or probes can be ligated using the splints (e.g., DNA) as a template. In some embodiments, a 3′ end and a 5′ end of polynucleotides of the oligonucleotides can be ligated using the probes (e.g., DNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, each end of each of the plurality of oligonucleotides is ligated, with or without gap filling, either to another of the plurality of oligonucleotides or to the first probe. In some embodiments, each end of each of the plurality of oligonucleotides is ligated, with or without gap filing, to another of the plurality of oligonucleotides, to the first probe, or to the second probe. In some embodiments, the first and second probe are ligated, with or without gap filling, to one another. In some embodiments, each end of each of the plurality of oligonucleotides is ligated to another of the plurality of oligonucleotides.

In some embodiments, the ligation of each end of an oligonucleotide of the plurality of oligonucleotides is templated ligation. In some embodiments, the template is a splint of the one or more splints. In some embodiments, the template is the first or second probe.

In some embodiments, the ligation is selected from the group consisting of enzymatic ligation, chemical ligation, template dependent ligation, and/or template independent ligation. In any of the embodiments herein, the ligation can comprise using a ligase having an RNA-templated DNA ligase activity and/or an RNA-templated RNA ligase activity. In any of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments herein, the ligation can comprise using a PBCV-1 DNA ligase or variant or derivative thereof and/or a T4 RNA ligase 2 (T4 Rn12) or variant or derivative thereof.

In some embodiments, the method can further comprise prior to the ligating step, a step of removing probes, splints, and/or oligonucleotides that are not bound to the target nucleic acid from the biological sample, the probes, and/or the splints. In any of the embodiments herein, the method can further comprise prior to the ligating step, a step of removing probes, splints, and/or oligonucleotides that are bound but comprise one or more mismatches in the hybridization regions. In any of the embodiments herein, the method can further comprise prior to the ligating step, a step of allowing probes, splints, and/or oligonucleotides that are bound but comprise one or more mismatches to dissociate from the target nucleic acid, probes, and/or splints, while probes, splints, and/or oligonucleotides comprising no mismatch remain bound. In any of the embodiments herein, under the same conditions, the molecules comprising one or more mismatches can be less stably bound than the molecules comprising no mismatch between the corresponding hybridization regions (e.g., between HR2 and HR2′). In any of the embodiments herein, the method can comprise one or more stringency washes. For instance, one or more stringency washes can be used to remove probes, splints, and/or oligonucleotides that are not bound, and/or probes, splints, and/or oligonucleotides that are bound but comprise one or more mismatches.

Following formation of the circular probe, in some instances an amplification primer is added. In other instances, the amplification primer is added with the probes, splints, and/or oligonucleotides, e.g., a splint can be used as a primer. In some instances, the amplification primer, e.g., a splint of the one or more splints, may also be complementary to the target nucleic acid and the circular probe (e.g., a SNAIL probe). A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase. In some instances, the amplification reaction uses the target nucleic acid to prime the RCA reaction.

In some embodiments, a washing step is performed to remove any unbound probes, primers, etc. In some embodiments, the wash is a stringency wash. Washing steps can be performed at any point during the process to remove non-specifically bound probes, probes that have ligated, etc.

Upon addition of a DNA polymerase in the presence of appropriate dNTP precursors and other cofactors, the amplification primer is elongated by replication of multiple copies of the template (e.g., a concatemer of the template, e.g., the circular probe, is generated). This amplification product can be detected using, e.g., the secondary and higher order probes and detection oligonucleotides described herein. In any of the embodiments herein, a sequence in the circular probe or amplification product can be determined or otherwise analyzed, for example by using detectably labeled probes and imaging. The sequencing or analysis of the circular probes or amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In some embodiments, upon addition of a DNA polymerase in the presence of appropriate dNTP precursors and other cofactors, a primer is elongated to produce multiple copies of the circular probe as a template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA), such as linear RCA, a branched RCA, a dendritic RCA, or any combination thereof, are described in, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801), all of which are herein incorporated by reference in their entireties. Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In any of the embodiments herein, the method can further comprise generating the product of the circular probe in situ in the biological sample. In any of the embodiments herein, the product can be generated using rolling circle amplification (RCA). In any of the embodiments herein, the RCA can comprise a linear RCA, a branched RCA, a dendritic RCA, or any combination thereof. In any of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, the RCA template (e.g., circular probe template formed using the probes, splints, and oligonucleotides as described herein) may be considered as a proxy, or a marker, for the target nucleic acid. In some embodiments, a RCA-based detection system is used, e.g., where the signal is provided by generating a RCP from a circular RCA template (e.g., generated as described herein), and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template (e.g., circular probe generated as described herein) generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence generated using a probe disclosed herein may be in a hybridization complex formed of a cellular nucleic acid in a sample and an exogenously added nucleic acid probe or probe set. The product can be detected by contacting with an exogenously added nucleic acid probe that may comprise an overhang that does not hybridize to the cellular nucleic acid or product generated using the probes, splints, and oligonucleotides described in Section III, but hybridizes to another probe (e.g., a detection oligonucleotide or a detectably labelled probe and/or a non-detectably labelled probe). In other examples, a product comprising a target sequence for a detection oligonucleotide disclosed herein may be an RCP generated using a circularizable probe or probe set which hybridizes to a cellular nucleic acid molecule (e.g., genomic DNA or mRNA) and the detection oligonucleotide may bind the RCP. In other examples, a product comprising a target sequence for a detection oligonucleotide disclosed herein (e.g., a detectably labelled probe and/or a non-detectably labelled probe may a probe hybridizing to an RCP. The probe may comprise an overhang that does not hybridize to the RCP but hybridizes to another probe (e.g., a detection oligonucleotide or a detectably labelled probe and/or a non-detectably labelled probe). The probe may be optionally ligated to a cellular nucleic acid molecule or another probe, e.g., an anchor probe that hybridizes to the RCP.

In any of the embodiments herein, the product can be immobilized in the biological sample. In any of the embodiments herein, the product can be crosslinked to one or more other molecules in the biological sample. Any suitable methods for tethering and immobilization may be used e.g., any described in Section II. In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833, US 2017/0219465, U.S. Pat. Nos. 10,138,509, 10,494,662, 11,078,520, 11,299,767, 10,266,888, 11,118,220, US 2021/0363579, and US 2021/0215581, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In any of the embodiments herein, the method can further comprise imaging the biological sample to detect the circular probe or product thereof. In any of the embodiments herein, the imaging can comprise detecting a signal associated with a fluorescently labeled probe that directly or indirectly binds to the circular probe or a rolling circle amplification product of the circular probe. In any of the embodiments herein, a sequence of the circular probe or rolling circle amplification product can be analyzed in situ in the biological sample. In any of the embodiments herein, the sequence of the circular probe or rolling circle amplification product can be analyzed by sequential hybridization, sequencing by hybridization, sequencing by ligation, sequencing by synthesis, sequencing by binding, or a combination thereof.

In some cases, analysis is performed on one or more images captured, and may comprise processing the image(s) and/or quantifying signals observed. In some embodiments, images of signals from different fluorescent channels and/or detectable probe hybridization (and optionally ligation) cycles can be compared and analyzed. In some embodiments, images of signals (or absence thereof) at a particular location in a sample from different fluorescent channels and/or sequential detectable probe hybridization (and optionally ligation) cycles can be aligned to analyze an analyte at the location. For instance, a particular location in a sample can be tracked and signal spots from sequential hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In any of the embodiments herein, the sequence of the circular probe or rolling circle amplification product can comprise one or more barcode sequences or complements thereof. In any of the embodiments herein, the one or more barcode sequences can comprise a barcode sequence corresponding to the target nucleic acid. In any of the embodiments herein, the one or more barcode sequences can comprise a barcode sequence corresponding to the sequence of interest, such as variant(s) of a single nucleotide of interest.

In any of the embodiments herein, the detecting step can comprise contacting the biological sample with one or more detectably-labeled probes (e.g., detection oligonucleotide) that directly or indirectly hybridize to the circular probe or rolling circle amplification product, and dehybridizing the one or more detectably-labeled probes from the circular probe or rolling circle amplification product. In any of the embodiments herein, the contacting and dehybridizing steps can be repeated with the one or more detectably-labeled probes and/or one or more other detectably-labeled probes that directly or indirectly hybridize to the circular probe or rolling circle amplification product.

In any of the embodiments herein, the detecting step can comprise contacting the biological sample with one or more intermediate probes that directly or indirectly hybridize to the circular probe or rolling circle amplification product, wherein the one or more intermediate probes are detectable using one or more detectably-labeled probes (e.g., detection oligonucleotides). In any of the embodiments herein, the detecting step can further comprise dehybridizing the one or more intermediate probes and/or the one or more detectably-labeled probes from the circular probe or rolling circle amplification product. In any of the embodiments herein, the contacting and dehybridizing steps can be repeated with the one or more intermediate probes, the one or more detectably-labeled probes, one or more other intermediate probes, and/or one or more other detectably-labeled probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple primary probes, and optionally multiple secondary probes hybridizing to the primary barcodes (or complementary sequences thereof) are all hybridized at once, followed by sequential secondary barcode detection and decoding of the signals. In some embodiments, detection of barcodes or subsequences of the barcode can occur in a cyclic manner.

In some embodiments, provided herein is a multiplexed assay where multiple probes are used to detect multiple target nucleic acids and/or one or more sequences of the same target nucleic acid molecule simultaneously.

In some embodiments, one or more detections of one or more target nucleic acids may occur simultaneously. In some embodiments, one or more detections of one or more target nucleic acids or one or more sequences may occur sequentially. In some embodiments, multiple probes of the same probe design are used to detect one or more target nucleic acids, using different barcodes associated with each region of interest. In some embodiments, multiple probes of different probe design are used to detect one or more target nucleic acids, using different barcodes (e.g., each barcode associated with a target nucleic acid or sequence thereof). In some embodiments, one or more target nucleic sequences that are localized on the same molecule (e.g., RNA or DNA) can be probed. In alternative embodiments, the one or more single nucleotides of interest are localized on different molecules.

In some embodiments, the circular probe includes 1, 2, 3, 4, 5, 6, 7, 8, 9, 10, 11, 12, 13, 14, 15 or more, 20 or more, 32 or more, 40 or more, or 50 or more barcode sequences. In some embodiments, the circular probe includes multiple copies of the same barcode sequence. In some embodiments, the circular probe contains only one distinct barcode sequence. In some embodiments, the circular probe includes multiple distinct barcode sequences.

In some embodiments, the number of distinct barcode sequences in a population of circular probes is less than the number of distinct targets (e.g., nucleic acid analytes and/or protein analytes) of the circular probes, and yet the distinct targets may still be uniquely identified from one another, e.g., by encoding a probe with a different combination of barcode sequences. However, not all possible combinations of a given set of barcode sequences need be used. For instance, each circular probe may contain 1, 2, 3, 4, 5, 6, 7, 8, 9, 10, 11, 12, 13, 14, 15, etc. or more barcode sequences. In some embodiments, a population of circular probes may each contain the same number of barcode sequences, although in other cases, there may be different numbers of barcode sequences present on the various circular probes. In some embodiments, the barcode sequences or any subset thereof in the population of circular probes can be independently and/or combinatorially detected and/or decoded.

As an illustrative example, a first circular probe may contain a first target-binding sequence, a first barcode sequence, and a second barcode sequence, while a second, different circular probe may contain a second target-binding sequence (that is different from the first target-binding sequence in the first circular probe), the same first barcode sequence as in the first circular probe, but a third barcode sequence instead of the second barcode sequence. Such probes may thereby be distinguished by determining the various barcode sequence combinations present or associated with a given circular probe at a given location in a sample.

A method disclosed herein is exemplified in FIG. 1 and FIG. 4. In some embodiments, the target nucleic acid is contacted with a first probe, one or more splints, and a plurality of oligonucleotides. The target nucleic acid can be a target molecule contained in a sample or an intermediate probe that is bound to the target molecule. In some embodiments, the first probe includes hybridization region HR2 that hybridizes to hybridization region HR2′ of the target nucleic acid. In some embodiments, the first probe further includes hybridization regions HR1 and HR3. In some embodiments, HR1 and HR3 do not hybridize to the target nucleic acid. In some embodiments, HR2 is between HR1 and HR3. In some embodiments, the first probe includes, from 5′ to 3′, hybridization regions HR1, HR2, and HR3. In some embodiments, the first probe includes, from 3′ to 5′, hybridization regions HR1, HR2, and HR3.

In some embodiments, the one or more splints are any as described in Section III. In some embodiments, the one or more splints include hybridization regions HR1′ and HR3′ that hybridize to HR1 and HR3, respectively. In some embodiments, the plurality of oligonucleotides are any as described in Section III. In some embodiments, the one or more splints include a plurality of hybridization regions that each hybridize to one or more of the plurality of oligonucleotides, e.g., to the ends of one or more of the plurality of oligonucleotides.

In some embodiments, the method further includes ligating the first probe to the plurality of oligonucleotides and the plurality of oligonucleotides to one another in order to form a circular probe that is hybridized to the target nucleic acid. In some embodiments, HR1 of the first probe is ligated to a first adjacent oligonucleotide. In some embodiments, HR3 of the first probe is ligated to a second adjacent oligonucleotide. In some embodiments, adjacent oligonucleotides are ligated to one another.

In some embodiments, the ligating is performed using the one or more splints as templates. In some embodiments, the one or more splints include DNA molecules, and the ligation includes DNA-templated ligation. In some embodiments, the one or more splints include RNA molecules, and the ligation includes RNA-templated ligation.

In some embodiments, the sequence length of the circular probe is greater than the combined length of HR2′ and the regions of the one or more splints that hybridize to the plurality of oligonucleotides. In some aspects, this increase is due at least in part to the inclusion of a loop sequence in one or more oligonucleotides that does not hybridize to the one or more splints, target nucleic acid, or first probe.

In some embodiments, the method further includes forming an amplification product using the circular probe as a template. In some embodiments, a splint is used as a primer for forming the amplification product, for instance as depicted in FIG. 1. In other embodiments, such as that depicted in FIG. 4, a separate primer for forming the amplification product is added.

In some embodiments as exemplified in FIG. 2 and FIG. 5, the target nucleic acid is contacted with a first probe, a second probe, one or more splints, and a plurality of oligonucleotides. The target nucleic acid can be a target molecule contained in a sample or an intermediate probe that is bound to the target molecule.

In some embodiments, the target nucleic acid includes hybridization regions HR2a′ and HR2b′. In some embodiments, HR2a′ and HR2b′ are adjacent to one another. In some embodiments, the target nucleic acid is contacted with a first probe and a second probe that hybridize to HR2a′ and HR2b′, respectively. In some embodiments, the first probe includes hybridization region HR2a that hybridizes to HR2a′. In some embodiments, the first probe also includes hybridization region HR1. In some embodiments, HR1 does not hybridize to the target nucleic acid. In some embodiments, the second probe includes hybridization region HR2b that hybridizes to HR2b′. In some embodiments, the second probe also includes hybridization region HR3. In some embodiments, HR3 does not hybridize to the target nucleic acid.

In some embodiments, the one or more splints are any as described in Section III. In some embodiments, the one or more splints include hybridization regions HR1′ and HR3′ that hybridize to HR1 and HR3, respectively. In some embodiments, the plurality of oligonucleotides are any as described in Section III. In some embodiments, the one or more splints include a plurality of hybridization regions that each hybridize to one or more of the plurality of oligonucleotides, e.g., to the ends of one or more of the plurality of oligonucleotides.

In some embodiments, the method further includes ligating the first probe and the second probe to the plurality of oligonucleotides and the plurality of oligonucleotides to one another in order to form a circular probe that is hybridized to the target nucleic acid. In some embodiments, HR1 of the first probe is ligated to a first adjacent oligonucleotide. In some embodiments, HR3 of the second probe is ligated to a second adjacent oligonucleotide. In some embodiments, adjacent oligonucleotides are ligated to one another. In some embodiments, the ligating is performed using the one or more splints as templates. In some embodiments, the one or more splints include DNA molecules, and the ligation includes DNA-templated ligation. In some embodiments, the one or more splints include RNA molecules, and the ligation includes RNA-templated ligation.

In some embodiments, the method further includes ligating the first probe and the second probe to one another. In some embodiments, HR2a is ligated to HR2b. In some embodiments, the ligating is performed using the target nucleic acid as a template. In some embodiments, the target nucleic acid is a DNA molecule, and the ligation includes DNA-templated ligation. In some embodiments, the target nucleic acid is an RNA molecule, and the ligation includes RNA-templated ligation.

In some embodiments, the ligating of HR1 to a first adjacent oligonucleotide, adjacent oligonucleotides to each other, and HR3 to a second adjacent oligonucleotide is performed simultaneously with the ligating of HR2a to HR2b. In some embodiments, the ligating of HR1 to a first adjacent oligonucleotide, adjacent oligonucleotides to each other, and HR3 to a second adjacent oligonucleotide is performed sequentially with the ligating of HR2a to HR2b. In some embodiments, the ligating of HR1 to a first adjacent oligonucleotide, adjacent oligonucleotides to each other, and HR3 to a second adjacent oligonucleotide is performed before the ligating of HR2a to HR2b. In some embodiments, the ligating of HR1 to a first adjacent oligonucleotide, adjacent oligonucleotides to each other, and HR3 to a second adjacent oligonucleotide is performed after the ligating of HR2a to HR2b.

In some embodiments, the ligating of HR2a to HR2b is without gap filling prior to ligation. In some embodiments, the ligating of HR2a to HR2b includes gap-filling using the target nucleic acid as a template. The gap may be 1, 2, 3, 4, 5, or more nucleotides. In some embodiments, the circular probe includes a gap-filled sequence. In some embodiments, the method further includes sequencing the gap-filled sequence.

In some embodiments, the sequence length of the circular probe is greater than the combined length of HR2a′, HR2b, and the regions of the one or more splints that hybridize to the plurality of oligonucleotides. In some aspects, this increase is due at least in part to the inclusion of a loop sequence in one or more oligonucleotides that does not hybridize to the one or more splints, target nucleic acid, first probe, or second probe.

In some embodiments, the method further includes forming an amplification product using the circular probe as a template. In some embodiments, a splint is used as a primer for forming the amplification product, for instance as depicted in FIG. 5. In other embodiments, such as that depicted in FIG. 2, a separate primer for forming the amplification product is added.

Another method disclosed herein is exemplified in FIG. 3. In some embodiments, the target nucleic acid is contacted with a first probe, a second probe, and a plurality of oligonucleotides. In some embodiments, the first and second probe hybridize to a first and second nucleic acid strand, respectively. The first and second nucleic acid strands can independently be a target molecule contained in the sample or an intermediate probe that is bound to the target molecule.

In some embodiments, the first and second nucleic acid strands are in proximity to one another. In some embodiments, the circular probe is formed only if the first and second nucleic acid strands are in proximity to one another. Thus, in some aspects, detection of the circular probe or an amplification product thereof can be used to detect two nucleic acid strands in proximity to one another. In some embodiments, the method allows for spatial analysis of nucleic acid strands contained in a sample. In some embodiments, the first and second nucleic acid strands are in close enough proximity to one another such that an oligonucleotide of the plurality of oligonucleotides hybridizes to both the first and second probes. In some embodiments, one end of the oligonucleotide hybridizes to the first nucleic acid strand, and the other end of the oligonucleotide hybridizes to the second nucleic acid strand. In some embodiments, a second oligonucleotide of the plurality of oligonucleotides hybridizes to both the first and second probes. In some embodiments, one end of the second oligonucleotide hybridizes to the first nucleic acid strand, and the other end of the second oligonucleotide hybridizes to the second nucleic acid strand.

In some embodiments, the plurality of oligonucleotides is any as described in Section III. In some embodiments, the first and second probes include a plurality of hybridization regions that each hybridize to one or more of the plurality of oligonucleotides, e.g., to the ends of one or more of the plurality of oligonucleotides.

In some embodiments, the method further includes ligating the plurality of oligonucleotides to one another in order to form a circular probe that is hybridized to the first and second probes. In some embodiments, adjacent oligonucleotides are ligated to one another. In some embodiments, the ligating is performed using the first and second probes as templates. In some embodiments, the first and second probes include DNA molecules, and the ligation includes DNA-templated ligation. In some embodiments, the first and second probes include RNA molecules, and the ligation includes RNA-templated ligation.

In some embodiments, the sequence length of the circular probe is greater than the combined length of the regions of the first and second probes that hybridize to the plurality of oligonucleotides. In some aspects, this increase is due at least in part to the inclusion of a loop sequence in one or more oligonucleotides that does not hybridize to the first probe, second probe, first nucleic acid strand, or second nucleic acid strand.

In some embodiments of any of the methods disclosed herein, the method further includes forming an amplification product using the circular probe as a template. In some embodiments, the amplification product is formed prior to steps of detecting a sequence in the amplification product. In other embodiments, a sequence is detected in the circular probe, e.g., without or prior to steps of forming an amplification product. For instance, in some embodiments where multiple copies of the same barcode sequence are present in the circular probe, amplification may not be necessary in order to detect the barcode sequence.

In some embodiments, the amplification product is performed using rolling circle amplification (RCA). In some embodiments, the RCA comprises a linear RCA. In some embodiments, the RCA comprises a branched RCA. In some embodiments, the RCA comprises a dendritic RCA. In some embodiments, the RCA comprises any combination of the foregoing. Any suitable methods and conditions for hybridization of probes, ligation, amplification, and detection may be used, e.g., as described in Sections

In some aspects, the provided methods involve analyzing, e.g., detecting or determining, one or more sequences present in the polynucleotides and/or in an amplification product, such as in a circular probe and/or an amplification product of the circular probe, which comprises one or more barcode sequences. In some embodiments, the analysis comprises determining the sequence of all or a portion of the circular probe and/or the amplification product. In some embodiments, the analysis comprises detecting a sequence present in the circular probe and/or the amplification product. In some embodiments, the sequence of all or a portion of the circular probe and/or the amplification product is indicative of the identity of a region (e.g., a single nucleotide) of interest in a target nucleic acid. In some embodiments, the analysis can be used to correlate a sequence detected in an amplification product to a circular probe (e.g., via a barcode). In some embodiments, the detection of a sequence in a circular probe and/or an amplification product can provide information regarding the location and/or identity of an associated target nucleic acid (e.g., hybridized by the circular probe) in a sample. In some embodiments, the circular probe includes a number of copies of the barcode sequence(s) that is sufficient for detection without amplification. In some embodiments, due to amplification of one or more polynucleotides (e.g., a circular probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of the circular probe.

In some aspects, the provided methods involve analyzing, e.g., detecting or determining, one or more sequences present in the polynucleotides and/or in a product or derivative thereof, such as in an amplified circular probe. In some embodiments, the circular probe or product thereof can be detected by providing detection probes, such as probes for performing a chain reaction that forms an amplification product, e.g., HCR. In some embodiments, the amplification is non-enzymatic. In some embodiments, the analysis comprises determining the sequence of all or a portion of the circular probe or amplification product. In some embodiments, the analysis comprises detecting a sequence present in the circular probe or amplification product. In some embodiments, the sequence of all or a portion of the circular probe or amplification product is indicative of the identity of a region of interest in a target nucleic acid. In other embodiments, the provided methods involve analyzing, e.g., detecting or determining, one or more sequences present in the polynucleotide probes (e.g., a barcode sequence present in a circular probe or product thereof).

In some embodiments, the methods comprise sequencing all or a portion of the circular probe or amplification product, such as one or more barcode sequences present in the circular probe or amplification product. In some embodiments, the analysis and/or sequence determination comprises sequencing all or a portion of the amplification product or the circular probe(s) and/or in situ hybridization to the amplification product or the circular probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the circular probe or amplification product a detection oligonucleotide labeled with a fluorophore, an isotope, a mass tag, or a combination thereof. In some embodiments, the detection or determination comprises imaging the circular probe or amplification product. In some embodiments, the target nucleic acid is an mRNA in a tissue sample, and the detection or determination is performed when the target nucleic acid and/or the amplification product is in situ in the tissue sample.

In some aspects, the provided methods involve analyzing, e.g., detecting or determining, one or more sequences (e.g., any of the barcodes described herein) present in the primary and second probes described herein and/or in a product or derivative thereof. In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the contents of which is incorporated herein by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the contents of which is incorporated herein by reference in its entirety), e.g., primer exchange reactions such as signal amplification by exchange reaction (SABER) or SABER with DNA-Exchange (Exchange-SABER), or any combination thereof. In some embodiments, non-enzymatic signal amplification may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401), all of which are incorporated herein by reference in their entireties. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., US 2022/0064697, the contents of which is incorporated herein by reference in its entirety), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes an assembly for branched signal amplification. In some embodiments, the assembly complex comprises an amplifier hybridized directly or indirectly (via one or more oligonucleotides) to a sequence (e.g., any of the barcodes described herein) present in the primary and second probes described herein and/or in a product or derivative thereof. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the sequences (e.g., any of the barcodes described herein) present in the primary and second probes described herein and/or in a product or derivative thereof can be detected in combination with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, the sample may be contacted with a plurality of concatemer primers and a plurality of labeled probes. see e.g., U.S. Pat. Pub. No. US20190106733, the contents of which is incorporated herein by reference in its entirety, for exemplary molecules and PER reaction components.

In some aspects, the provided methods comprise imaging the circular probe or amplification product (e.g., amplicon) and/or one or more portions of the polynucleotides, for example, via binding of the detection probe (e.g., detection oligonucleotide) and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more circular probe(s) and/or amplification products (e.g., amplicon) described herein. In some embodiments, the methods involve incubating the detectable probe containing the detectable label with the sample, washing unbound detectable probe, and detecting the label, e.g., by imaging. In some embodiments, a detectable probe may bind directly or indirectly to the circular probe and/or amplification products.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, the fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345, the content of which is herein incorporated by reference in its entirety.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, the content of which is herein incorporated by reference in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, the antibody is an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537, 4,849,336, 5,073,562, and PCT publication WO 91/17160, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, all of which are herein incorporated by reference in their entireties. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing comprise, but are not limited to, STARmap (described for example in Wang et al., (2018) Science, 361(6499) 5691, the content of which is herein incorporated by reference in its entirety), MERFISH (described for example in Moffitt, (2016) Methods in Enzymology, 572, 1-49, the content of which is herein incorporated by reference in its entirety), hybridization-based in situ sequencing (HybISS) (described for example in Gyllborg et al., Nucleic Acids Res (2020) 48(19):e112, the content of which is herein incorporated by reference in its entirety), and FISSEQ (described for example in US 2019/0032121, the content of which is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, the barcodes of the probes (e.g., the circular probe) or complements or products thereof are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; US 2017/0220733 A1, US 2020/0080139, U.S. Pat. No. 11,098,303, US 2019/0233812, and US 2022/0025442, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### V. Generation of Subsequent Circular Probes

In some embodiments, the provided methods involve forming one or more subsequent circular probes, for instance to perform multiplexed detection of multiple targets or regions of interest. In some embodiments, the circular probe formed according to the provided methods includes one or more cleavage sites, and a subsequent probe is formed by cleaving the one or more cleavage sites. In some embodiments, the subsequent probe is or remains hybridized to the target nucleic acid. In some embodiments, the one or more cleavage sites are comprised in sequences of the circular probe that do not hybridize to the target nucleic acid. In some embodiments, the cleaving is performed after analysis of the circular probe or of sequences, e.g., barcode sequences, contained in the circular probe, for example any analysis described in Section IV. In some embodiments, the subsequent probe is a U-probe. In some embodiments, the ends of the subsequent probe are not hybridized to the target nucleic acid.

In some embodiments, the subsequent probe formed by cleaving the circular probe includes all of the oligonucleotides used to form the original circular probe. In some embodiments, the subsequent probe includes all barcode sequences contained in the original circular probe. In some embodiments, the subsequent probe is formed by cleaving a single cleavage site in the circular probe.

In some embodiments, the subsequent probe formed by cleaving the circular probe includes a subset of the oligonucleotides used to form the original circular probe. In some embodiments, the subsequent probe includes a subset of the barcode sequences contained in the original circular probe. In some embodiments, the subsequent probe is formed by cleaving at least two cleavage sites in the circular probe. In some embodiments, the oligonucleotides and/or barcode sequences are removed from the subsequent probe by the cleaving.

In some embodiments, a cleavage site includes a disulfide bond. A reducing agent can be added to break the disulfide bond, resulting in cleavage of the circular probe. As another example, heating can also result in degradation of the cleavage site and cleavage of the circular probe. In some embodiments, laser radiation is used to heat and degrade cleavage sites at specific locations. In some embodiments, the cleavage site includes a photo-sensitive chemical bond (e.g., a chemical bond that dissociates when exposed to light such as ultraviolet light).

Other examples of cleavage sites include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

In some embodiments, the cleavage site includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage site can include a restriction endonuclease (restriction enzyme) recognition sequence. In some aspects, restriction enzymes cut double-stranded or single stranded DNA at specific recognition nucleotide sequences known as restriction sites. In some embodiments, a rare-cutting restriction enzyme, e.g., enzymes with a long recognition site (at least 8 base pairs in length), is used to reduce the possibility of cleaving elsewhere in the circular probe.

In some embodiments, the cleavage site includes a poly(U) sequence which can be cleaved by a mixture of Uracil DNA glycosylase (UDG) and the DNA glycosylase-lyase Endonuclease VIII, commercially known as the USER™ enzyme.

In some embodiments, following the cleaving, the subsequent probe is contacted with one or more additional splints and one or more additional oligonucleotides. The additional splints and oligonucleotides can be any of the splints and oligonucleotides, respectively, that are described herein. The subsequent probe can be contacted with the additional splints and oligonucleotides simultaneously or sequentially in either order.

In some embodiments, the additional splints hybridize to sequences at the ends of the subsequent probe. In some embodiments, the additional oligonucleotides hybridize to the additional splints. In some embodiments, sequences at the ends of the additional oligonucleotides hybridize to the additional splints, and sequences between the ends form loops upon hybridization. In some embodiments, the loop sequences do not hybridize to other polynucleotides. In some embodiments, the loop sequences include barcode sequences.

In some embodiments, the one or more additional splints include at least 1, 2, 3, 4, 5, 6, 7, 8, 9, or 10 splints. In some embodiments, the one or more additional splints include up to 10 splints. In some embodiments, the one or more additional splints include between or between about 1 and 10 splints, 1 and 9 splints, 1 and 8 splints, 1 and 7 splints, 1 and 6 splints, 1 and 5 splints, 1 and 4 splints, 1 and 3 splints, 2 and 10 splints, 2 and 9 splints, 2 and 8 splints, 2 and 7 splints, 2 and 6 splints, 2 and 5 splints, 2 and 4 splints, 3 and 10 splints, 3 and 9 splints, 3 and 8 splints, 3 and 7 splints, 3 and 6 splints, 3 and 5 splints, 4 and 10 splints, 4 and 9 splints, 4 and 8 splints, 4 and 7 splints, 4 and 6 splints, 5 and 10 splints, 5 and 9 splints, 5 and 8 splints, 5 and 7 splints, 6 and 10 splints, 6 and 9 splints, 6 and 8 splints, 7 and 10 splints, 7 and 9 splints, and 8 and 10 splints, each inclusive. In some embodiments, the one or more additional splints include between or between about 1 and 10 splints, inclusive, for instance 1, 2, 3, 4, 5, 6, 7, 8, 9, or 10 splints.

In some embodiments, the one or more additional oligonucleotides include at least 1, 2, 3, 4, 5, 6, 7, 8, 9, 10, 11, 12, 13, 14, 15, 16, 17, 18, 19, or 20 oligonucleotides. In some embodiments, the one or more additional oligonucleotides includes up to 20 oligonucleotides. In some embodiments, the one or more additional oligonucleotides include between or between about 1 and 20 oligonucleotides, 1 and 18 oligonucleotides, 1 and 16 oligonucleotides, 1 and 14 oligonucleotides, 1 and 12 oligonucleotides, 1 and 10 oligonucleotides, 1 and 8 oligonucleotides, 1 and 6 oligonucleotides, 1 and 4 oligonucleotides, 2 and 20 oligonucleotides, 2 and 18 oligonucleotides, 2 and 16 oligonucleotides, 2 and 14 oligonucleotides, 2 and 12 oligonucleotides, 2 and 10 oligonucleotides, 2 and 8 oligonucleotides, 2 and 6 oligonucleotides, 2 and 4 oligonucleotides, 4 and 20 oligonucleotides, 4 and 18 oligonucleotides, 4 and 16 oligonucleotides, 4 and 14 oligonucleotides, 4 and 12 oligonucleotides, 4 and 10 oligonucleotides, 4 and 8 oligonucleotides, 4 and 6 oligonucleotides, 6 and 20 oligonucleotides, 6 and 18 oligonucleotides, 6 and 16 oligonucleotides, 6 and 14 oligonucleotides, 6 and 12 oligonucleotides, 6 and 10 oligonucleotides, 6 and 8 oligonucleotides, 8 and 20 oligonucleotides, 8 and 18 oligonucleotides, 8 and 16 oligonucleotides, 8 and 14 oligonucleotides, 8 and 12 oligonucleotides, 8 and 10 oligonucleotides, 10 and 20 oligonucleotides, 10 and 18 oligonucleotides, 10 and 16 oligonucleotides, 10 and 14 oligonucleotides, 10 and 12 oligonucleotides, 12 and 20 oligonucleotides, 12 and 18 oligonucleotides, 12 and 16 oligonucleotides, 12 and 14 oligonucleotides, 14 and 20 oligonucleotides, 14 and 18 oligonucleotides, 14 and 16 oligonucleotides, 16 and 20 oligonucleotides, 16 and 18 oligonucleotides, or 18 and 20 oligonucleotides, each inclusive. In some embodiments, the one or more additional oligonucleotides include between or between about 1 and 20 oligonucleotides, for instance 1, 2, 3, 4, 5, 6, 7, 8, 9, 10, 11, 12, 13, 14, 15, 16, 17, 18, 19, or 20 oligonucleotides.

In some embodiments, at least one of the one or more additional oligonucleotides includes a barcode sequence. In some embodiments, at least 1, 2, 3, 4, 5, 6, 7, 8, 9, 10, 11, 12, 13, 14, 15, 16, 17, 18, 19, or 20 of the one or more additional oligonucleotides includes a barcode sequence. In some embodiments, each of the one or more additional oligonucleotides includes a barcode sequence. In some embodiments, the barcode sequence is contained within the loop sequence of the additional oligonucleotide.

In some embodiments, the additional oligonucleotides are ligated to each other and to the ends of the subsequent probe in order to form a subsequent circular probe. The ligation can be performed according to any of the methods described herein. In some embodiments, the additional splints are used as templates for the ligation. In some embodiments, each end of an additional oligonucleotide of the additional oligonucleotides is ligated either to the subsequent probe or to another of the additional oligonucleotides.

In some aspects, the provided methods involve analyzing, e.g., detecting or determining, one or more sequences present in the subsequent circular probe and/or in an amplification product thereof. The amplifying and/or analyzing of the subsequent circular probe can be performed according to any of the methods described herein.

In some embodiments, the provided methods involve performing multiple rounds of cleaving a circular probe to form a subsequent probe, adding and ligating additional splints and oligonucleotides to form a subsequent circular probe, and analyzing the subsequent circular probe or an amplification product thereof. In some embodiments, at least 1, 2, 3, 4, 5, 6, 7, 8, 9, or 10 rounds are performed. In some embodiments, 1, 2, 3, 4, 5, 6, 7, 8, 9, or 10 rounds are performed. In some embodiments, different sets of additional splints and oligonucleotides are added across rounds in order to generate different circular probes, for instance in order to perform multiplexed detection of multiple target nucleic acids of interest.

### VI. Compositions and Kits

In some aspects, disclosed herein is a composition that comprises a complex comprising a target nucleic acid and one or more probes, splints, and oligonucleotides. In some embodiments, the composition comprises a target nucleic acid, a first probe, one or more splints, and a plurality of oligonucleotides. In some embodiments, the composition comprises a target nucleic acid, a first and second probe, one or more splints, and a plurality of oligonucleotides. In some embodiments, the composition comprises a first and second nucleic acid strand, a first and second probe, and a plurality of oligonucleotides. In some embodiments, the probes, splints, and oligonucleotides are any as described herein. In some embodiments, the target nucleic acid is any as described herein. In some embodiments, the first and second nucleic acid strands are any as described herein. In some embodiments, the composition comprises a target nucleic acid or multiple target nucleic acids and a circular probe formed using any of the probes, splints, and oligonucleotides described herein. In some embodiments, the composition comprises a set of probe(s), splints, and oligonucleotides, e.g., any as described herein, for each target nucleic acid. In some aspects, the compositions comprises multiple sets of probe(s), splints, and oligonucleotides, e.g., any as described herein, wherein each set is designed for a target nucleic acid. In some embodiments, the composition further includes a primer for amplification of the circular probe. In some embodiments, the composition comprises a target nucleic acid or multiple target nucleic acids and an amplification product of the circular probe.

Also provided herein are kits comprising one or more polynucleotides, including any of the polynucleotides as described in Section III, and reagents for performing the methods provided herein, for example reagents required for one or more steps comprising hybridization, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the kit further comprises a ligase, for instance for forming a ligated circular probe from the probes and/or oligonucleotides. In some embodiments, the ligase has DNA-splinted DNA ligase activity. In some embodiments, the kit further comprises a polymerase, for instance for performing amplification of the circular probe. In some embodiments, the polymerase is capable of using the ligated circular probe as a template for amplification. In some embodiments, the kit further comprises a primer for amplification.

In some aspects, provided herein is a kit for generating a circular probe, comprising: a first probe, one or more splints, and a plurality of oligonucleotides, wherein: (i) the first probe comprises hybridization regions HR1, HR2, and HR3, wherein HR2 hybridizes to hybridization region HR2′ of a target nucleic acid; and (ii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint to complementary sequences at the ends of an oligonucleotide, a sequence of the oligonucleotide between the ends forms a loop, and the sequence of the loop does not hybridize to the one or more splints.

In some aspects, provided herein is a kit for generating a circular probe, comprising: a first probe, a second probe, one or more splints, and a plurality of oligonucleotides, wherein: (i) the first probe comprises hybridization regions HR1 and HR2a; (ii) the second probe comprises hybridization regions HR2b and HR3; wherein HR2a and HR2b hybridize to adjacent hybridization regions HR2a′ and HR2b′, respectively, of a target nucleic acid; and (iii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3; wherein upon hybridization of two regions of a splint to complementary sequences at the ends of an oligonucleotide, a sequence of the oligonucleotide between the ends forms a loop, and the sequence of the loop does not hybridize to the one or more splints.

In some aspects, provided herein is a kit for generating a circular probe, comprising: a first probe, a second probe, and a plurality of oligonucleotides, wherein: (i) the first probe hybridizes to a first nucleic acid strand in a sample; (ii) the second probe hybridizes to a second nucleic acid strand in the sample; wherein the first and second nucleic acid strands are in proximity to one another; (iii) the first and second probes each comprise a plurality of hybridization regions that each hybridize to the ends of one or more of the plurality of oligonucleotides; wherein upon hybridization of two regions of a probe to complementary sequences at the ends of an oligonucleotide, a sequence of the oligonucleotide between the ends forms a loop, and the sequence of the loop does not hybridize to the first or second probe; and (iv) the first and second probes both hybridize to one or more of the plurality of oligonucleotides.

In some embodiments, the first probe is any as described herein. In some embodiments, the second probe is any as described herein. In some embodiments, the one or more splints is any as described herein. In some embodiments, the plurality of oligonucleotides is any as described herein.

In some embodiments, the kit further includes reagents for performing the methods provided herein, for example reagents required for one or more steps comprising hybridization, ligation, amplification, detection, sequencing, and/or sample preparation as described herein. In some embodiments, the kit further comprises a target nucleic acid, e.g., any as described herein. In some embodiments, any or all of the polynucleotides are DNA molecules. In some embodiments, the target nucleic acid is a messenger RNA molecule. In some embodiments, the kit further comprises a ligase, for instance for forming a ligated circular probe from the probes and/or oligonucleotides. In some embodiments, the ligase has DNA-splinted DNA ligase activity. In some embodiments, the kit further comprises a polymerase, for instance for performing amplification of the circular probe. In some embodiments, the polymerase is capable of using the ligated circular probe as a template for amplification. In some embodiments, the kit further comprises a primer for amplification.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, or reagents for additional assays.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions or sequences of interest in target nucleic acids.

In some embodiments, the region of interest comprises a single-nucleotide polymorphism (SNP). In some embodiments, the region of interest comprises a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example for characterization or assessment of particular cells or a tissue from a subject. Applications of the provided embodiments can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences (e.g., a constant region from each of two distinct capture probes) become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™ ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yekkow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, Mit® Tracker® Green, Mit® Tracker® Orange, Mit® Tracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and is not intended to limit the scope of the present disclosure.

### Example 1: Use of a First Probe, One or More Splints, and a Plurality of Oligonucleotides to Detect a Target Nucleic Acid

This example describes the use of a probe set comprising a first probe (e.g., U-probe), one or more splints, and a plurality of oligonucleotides for detecting a region of interest in a target nucleic acid. In this example, the probe set is used to generate a large circular probe. The circular probe can be detected, for instance by detection of any barcode sequences comprised therein, in order to detect the region of interest. Alternatively, the circular probe can be used as a template to form an amplification product that is detected in order to detect the region of interest.

A tissue sample is obtained and cryosectioned onto a glass slide for processing. The tissue is fixed by incubating in 3.7% paraformaldehyde (PFA). One or more washes is performed, and the tissue is then permeabilized. To prepare for probe hybridization, a wash buffer is added to the tissue section.

A mixture of the probe set is incubated with the thin tissue section sample and hybridization buffer for hybridization of the probe set to target nucleic acids (e.g., mRNAs) in the sample. The probe set comprises the first probe, one or more splints, and plurality of oligonucleotides depicted in FIG. 1 or FIG. 4. In some cases, the first probe is first contacted with the sample, and subsequently the one or more splints and plurality of oligonucleotides are added. The first probe comprises hybridization region HR2 that hybridizes to hybridization region HR2′ in the target nucleic acid. The ends of the first probe, which comprise hybridization regions HR1 and HR3, do not hybridize to the target nucleic acid. One of the splints (Splint 1 in FIG. 1 and FIG. 4) comprises hybridization region HR1′ that hybridizes to hybridization region HR1. Adjacent to hybridization region HR1′, this splint also comprises a sequence complementary to the end of one of the plurality of oligonucleotides. Another of the splints (Splint 3 in FIG. 1; Splint 2 in FIG. 4) comprises hybridization region HR3′ that hybridizes to hybridization region HR3. Adjacent to hybridization region HR3′, this splint also comprises a sequence complementary to the end of another of the plurality of oligonucleotides. The adjacent ends of splints hybridize to the same oligonucleotide of the plurality of oligonucleotides. The ends of each of the plurality of oligonucleotides hybridize to the one or more splints such that a loop that does not hybridize to the one or more splints forms in each of the plurality of oligonucleotides. In some aspects, this can allow for the inclusion of more oligonucleotides into a circularizable probe set that, following ligation, forms a large circular probe comprising the first probe and plurality of oligonucleotides.

Following hybridization, the first probe and plurality of oligonucleotides are hybridized such that they can be ligated to one another to form a large circular probe. The sample is then washed and incubated at room temperature with a T4 DNA ligase for ligation of the adjacent 5′ and 3′ ends of the plurality of oligonucleotides and first probe to one another to form circular probes. The one or more splints are used for templated ligation. Each end of each of the plurality of oligonucleotides is ligated either to another of the plurality of oligonucleotides or to the first probe.

Fluorescently labeled oligonucleotides (e.g., detection oligonucleotides) complementary to a portion of the circular probe, a barcode contained therein, or a secondary probe attached thereto are incubated with the sample. Multiple cycles of contacting the sample with probes and sequence determination (e.g., using in situ sequencing based on sequencing-by-ligation or sequencing-by-hybridization) can be performed. Fluorescent images can be obtained in each cycle, and one or more wash steps can be performed in a cycle or between cycles.

Optionally before the ligation step, and potentially before an optional amplification step, probe(s) that do not specifically hybridize to target nucleic acids in the sample can be disassociated from the target nucleic acids in the sample. The disassociation can comprise performing a stringent wash, e.g., a wash at a melting temperature that allows probes that are specifically hybridized to the target nucleic acid(s) to remain hybridized while probes comprising one or more mismatches are disassociated.

For an optional amplification step, a primer for amplification of the circular probe may be added. Alternatively, one of the splints (Splint 1 in FIG. 1) can be used as a primer for amplification of the circular probe. The sample is then incubated with a rolling-circle amplification (RCA) mixture containing a Phi29 DNA polymerase and dNTPs for RCA of the circular probes. Fluorescently labeled oligonucleotides complementary to a portion of the RCA product, a barcode contained therein, or a secondary probe attached thereto are incubated with the sample. Multiple cycles of contacting the sample with probes and sequence determination (e.g., using in situ sequencing based on sequencing-by-ligation or sequencing-by-hybridization) can be performed. Fluorescent images can be obtained in each cycle, and one or more wash steps can be performed in a cycle or between cycles.

### Example 2: Use of a First Probe, Second Probe, One or More Splints, and a Plurality of Oligonucleotides to Detect a Target Nucleic Acid

This example describes the use of a probe set comprising a first probe, a second probe, one or more splints, and a plurality of oligonucleotides for detecting a region of interest in a target nucleic acid. In this example, the probe set is used to generate a large circular probe. The circular probe can be detected, for instance by detection of any barcode sequences comprised therein, in order to detect the region of interest. Alternatively, the circular probe can be used as a template to form an amplification product that is detected in order to detect the region of interest.

A tissue sample is obtained and cryosectioned onto a glass slide for processing. The tissue is fixed by incubating in 3.7% paraformaldehyde (PFA). One or more washes is performed, and the tissue is then permeabilized. To prepare for probe hybridization, a wash buffer is added to the tissue section.

A mixture of the probe set is incubated with the thin tissue section sample and hybridization buffer for hybridization of the probe set to target nucleic acids (e.g., mRNAs) in the sample. The probe set comprises the first probe, second probe, one or more splints, and plurality of oligonucleotides depicted in FIG. 2 or FIG. 5. In some cases, the first probe and the second probe is first contacted with the sample, and subsequently the one or more splints and plurality of oligonucleotides are added. The first probe comprises hybridization region HR2a that hybridizes to hybridization region HR2a′ in the target nucleic acid. The first probe also comprises hybridization region HR1 that does not hybridize to the target nucleic acid. The second probe comprises hybridization region HR2b that hybridizes to hybridization region HR2b′ in the target nucleic acid. The second probe also comprises hybridization region HR3 that does not hybridize to the target nucleic acid. Hybridization regions HR2a′ and HR2b′ are adjacent to one another in the target nucleic acid.

One of the splints (Splint 1 in FIG. 2 and FIG. 5) comprises hybridization region HR1′ that hybridizes to hybridization region HR1. Adjacent to hybridization region HR1′, this splint also comprises a sequence complementary to the end of one of the plurality of oligonucleotides. Another of the splints (Splint 2 in FIG. 2; Splint 3 in FIG. 5) comprises hybridization region HR3′ that hybridizes to hybridization region HR3. Adjacent to hybridization region HR3′, this splint also comprises a sequence complementary to the end of another of the plurality of oligonucleotides. The adjacent ends of splints hybridize to the same oligonucleotide of the plurality of oligonucleotides. The ends of each of the plurality of oligonucleotides hybridize to the one or more splints such that a loop that does not hybridize to the one or more splints forms in each of the plurality of oligonucleotides. In some aspects, this can allow for the inclusion of more oligonucleotides into a circularizable probe set that, following ligation, forms a large circular probe comprising the first probe, second probe, and plurality of oligonucleotides.

Following hybridization, the first probe, second probe, and plurality of oligonucleotides are hybridized such that they can be ligated to one another to form a large circular probe. The sample is then washed and incubated at room temperature with a T4 DNA ligase for ligation of the adjacent 5′ and 3′ ends of the plurality of oligonucleotides, first probe, and second probe to one another to form circular probes. The one or more splints are used for templated ligation of the plurality of oligonucleotides to each other and to the first and second probes. The target nucleic acid is used for templated ligation of the first and second probes to each other. Each end of each of the plurality of oligonucleotides is ligated to another of the plurality of oligonucleotides, to the first probe, or to the second probe. The first and second probe are ligated to each other.

For an optional amplification step, a primer for amplification of the circular probe may be added. Alternatively, one of the splints (Splint 1 in FIG. 5) can be used as a primer for amplification of the circular probe. The sample is then incubated with a rolling-circle amplification (RCA) mixture containing a Phi29 DNA polymerase and dNTPs for RCA of the circular probes.

Detection of the circular probe or any products thereof is performed as substantially described in Example 1.

### Example 3: Use of a First Probe, Second Probe, and a Plurality of Oligonucleotides to Detect Nucleic Acid Strands in Proximity to Each Other

This example describes the use of a probe set comprising a first probe, a second probe, and a plurality of oligonucleotides for detecting two nucleic acid strands in proximity with one another. In this example, the probe set is used to generate a large circular probe. The circular probe can be detected, for instance by detection of any barcode sequences comprised therein, in order to detect the region of interest. Alternatively, the circular probe can be used as a template to form an amplification product that is detected in order to detect the region of interest.

A tissue sample is obtained and cryosectioned onto a glass slide for processing. The tissue is fixed by incubating in 3.7% paraformaldehyde (PFA). One or more washes is performed, and the tissue is then permeabilized. To prepare for probe hybridization, a wash buffer is added to the tissue section.

A mixture of the probe set is incubated with the thin tissue section sample and hybridization buffer for hybridization of the probe set to target nucleic acids (e.g., mRNAs) in the sample. The probe set comprises the first probe, second probe, and plurality of oligonucleotides depicted in FIG. 3. In some cases, the first probe and the second probe is first contacted with the sample, and subsequently the one or more splints and plurality of oligonucleotides are added. The first probe hybridizes to a first nucleic acid strand in the sample, and the second probe hybridizes to a second nucleic acid strand in the sample. The first and second nucleic acid strands are in close enough proximity to one another such that an oligonucleotide of the plurality of oligonucleotides can hybridize to both the first and second probes. The ends of each of the plurality of oligonucleotides hybridize to the first or second probe such that a loop that does not hybridize to the first or second probe forms in each of the plurality of oligonucleotides. In some aspects, this can allow for the inclusion of more oligonucleotides into a circularizable probe set that, following ligation, forms a large circular probe comprising the plurality of oligonucleotides.

Following hybridization, the first probe, second probe, and plurality of oligonucleotides are hybridized such that the plurality of oligonucleotides can be ligated to one another to form a large circular probe. The sample is then washed and incubated at room temperature with a T4 DNA ligase for ligation of the adjacent 5′ and 3′ ends of the plurality of oligonucleotides to form circular probes. The first and second probes are used for templated ligation of the plurality of oligonucleotides to each other. Each end of each of the plurality of oligonucleotides is ligated to another of the plurality of oligonucleotides.

Following circular probe generation, detection of the circular probe or any products thereof and any optional amplification is performed as substantially described in Example 1.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-4. (canceled)

5. A method for generating a circular probe, comprising:
(a) contacting a target nucleic acid with a target-binding probe or probe set, one or more splints, and a plurality of oligonucleotides, wherein:
(i) the target-binding probe or probe set comprises hybridization regions HR1, HR2, and HR3;
(ii) the target nucleic acid comprises a hybridization region HR2′ that hybridizes to HR2; and
(iii) the one or more splints comprise: a hybridization region HR1′ that hybridizes to HR1; a plurality of hybridization regions that each hybridize to the ends of one or more of the plurality of oligonucleotides; and a hybridization region HR3′ that hybridizes to HR3;
wherein upon hybridization of two regions of a splint of the one or more splints to complementary sequences at the ends of an oligonucleotide of the plurality of oligonucleotides, a sequence of the oligonucleotide between the ends forms a loop, and the sequence of the loop does not hybridize to the one or more splints; and
(b) ligating HR1 to a first adjacent oligonucleotide of the plurality of oligonucleotides, adjacent oligonucleotides of the plurality of oligonucleotides to one another, and HR3 to a second adjacent oligonucleotide of the plurality of oligonucleotides using the one or more splints as templates, thereby forming a circular probe that is hybridized to the target nucleic acid.

6. (canceled)

7. The method of claim 5, wherein the target-binding probe or probe set comprises a first probe that comprises hybridization regions HR1, HR2, and HR3.

8. (canceled)

9. The method of claim 5, wherein:
HR2′ is a split hybridization region comprising adjacent hybridization regions HR2a′ and HR2b′;
HR2 comprises hybridization regions HR2a and HR2b that hybridize to HR2a′ and HR2b′, respectively;
the target-binding probe or probe set comprises (i) a first probe that comprises HR1 and HR2a; and (ii) a second probe that comprises HR2b and HR3; and
HR2a is ligated to HR2b using the target nucleic acid as a template.

10. (canceled)

11. (canceled)

12. The method of claim 5, wherein the one or more splints comprise between or between about 1 and 10 splints.

13. The method of claim 5, wherein at least one of the one or more splints hybridizes to the ends of at least two oligonucleotides, wherein upon hybridization, a sequence between the ends of each of the at least two oligonucleotides forms a loop, and the sequence of each loop does not hybridize to the one or more splints.

14. The method of claim 5, wherein each of the one or more splints hybridizes to the ends of at least two oligonucleotides, wherein upon hybridization, a sequence between the ends of each of the at least two oligonucleotides forms a loop, and the sequence of each loop does not hybridize to the one or more splints.

15. The method of claim 5, wherein the one or more splints comprise at least two splints.

16. The method of claim 15, wherein adjacent splints of the at least two splints both hybridize to one of the plurality of oligonucleotides.

17. The method of claim 5, wherein the target nucleic acid is in a sample, and the contacting, hybridizing, and ligating are performed in situ.

18-22. (canceled)

23. A method for generating a circular probe, comprising:
(a) contacting a sample comprising a first nucleic acid strand and a second nucleic acid strand in proximity to one another with a first probe, a second probe, and a plurality of oligonucleotides, wherein:
(i) the first probe hybridizes to the first nucleic acid strand;
(ii) the second probe hybridizes to the second nucleic acid strand;
(iii) the first and second probes each comprise a plurality of hybridization regions that each hybridize to the ends of one or more of the plurality of oligonucleotides;
wherein upon hybridization of two regions of a probe of the first and second probes to complementary sequences at the ends of an oligonucleotide of the plurality of oligonucleotides, a sequence of the oligonucleotide between the ends forms a loop, and the sequence of the loop does not hybridize to the first or second probe; and
(iv) the first and second probes both hybridize to one or more of the plurality of oligonucleotides; and
(b) ligating adjacent oligonucleotides of the plurality of oligonucleotides to one another using the first and second probes as templates, thereby forming a circular probe that is hybridized to the first and second probes.

24. (canceled)

25. (canceled)

26. The method of claim 23, wherein one or both of the first and second probes hybridize to the ends of at least two oligonucleotides, wherein upon hybridization, a sequence between the ends of each of the at least two oligonucleotides forms a loop, and the sequence of each loop does not hybridize to the first or second probe.

27. The method of claim 23, wherein the first and second probes each hybridize to the ends of at least two oligonucleotides, wherein upon hybridization, a sequence between the ends of each of the at least two oligonucleotides forms a loop, and the sequence of each loop does not hybridize to the first or second probe.

28-30. (canceled)

31. The method of claim 5, wherein the plurality of oligonucleotides comprise between or between about 2 and 20 oligonucleotides.

32. The method of claim 5, wherein the loop of at least 1 of the plurality of oligonucleotides comprises a barcode sequence.

33. The method of claim 5, wherein each loop of the plurality of oligonucleotides comprises a barcode sequence.

34. (canceled)

35. The method of claim 5, further comprising detecting a sequence in the circular probe.

36. The method of claim 5, further comprising forming an amplification product using the circular probe as a template and detecting a sequence in the amplification product.

37. The method of claim 36, wherein one of the one or more splints is used as a primer for forming the amplification product.

38. (canceled)

39. (canceled)

40. The method of claim 5, wherein the circular probe comprises one or more cleavage sites, and the method further comprises:
(a) cleaving the one or more cleavage sites to form a subsequent probe;
(b) contacting the subsequent probe with one or more additional splints and one or more additional oligonucleotides, wherein:
the one or more additional splints comprise a plurality of hybridization regions that each hybridize to the ends of at least one of the one or more additional oligonucleotides, wherein a sequence between the ends forms a loop upon additional oligonucleotide-additional splint hybridization, and the loop does not hybridize to the one or more additional splints; and
the one or more additional splints hybridize to the ends of the subsequent probe; and
(c) ligating each end of the subsequent probe to an adjacent additional oligonucleotide of the one or more additional oligonucleotides using the one or more additional splints as templates, thereby forming a subsequent circular probe.

41-56. (canceled)

57. The method of claim 36, wherein the target nucleic acid is in a sample, and the detecting is performed in situ.

58-68. (canceled)
